(19) United States
(12) Patent Application Publication
(10) Pub. No.: US 2003/0198941 A1
(43) Pub. Date: Oct. 23, 2003

(54) Method for making an HIV vaccine

(76) Inventors: David I. Watkins, Arena, WI (US); Todd M. Allen, Madison, WI (US); David H. O'Connor, Madison, WI (US); Bianca R. Mothe, Madison, WI (US); Thorsten U. Vogel, Madison, WI (US); Helen Horton, Madison, WI (US)

Correspondence Address: QUARLES & BRADY LLP, 411 E. WISCONSIN AVENUE, SUITE 2040, MILWAUKEE, WI, 53202-4497, US

(21) Appl. No.: 09/829,228

(22) Filed: Apr. 9, 2001

Related U.S. Application Data
(60) Provisional application No. 60/196,412, filed on Apr. 12, 2000.

(51) Int. Cl. C12Q 1/70; A61K 39/21

(52) U.S. Cl. 435/5

(57) ABSTRACT

A method of identifying at least one CTL-inducing epitope from HIV protein is disclosed. In one embodiment, the method comprises the steps of (a) examining the nucleic acid sequence encoding at least one HIV protein from at least one HIV-infected patient, wherein the sequence encoding the expressed protein is examined in the first six months after infection, to identify at least one region of the HIV protein that is variable as compared to the sequence of the protein at an earlier time point in infection, wherein the variable region indicates a CTL-inducing epitope, and (b) confirming that an immune response directed against the CTL-inducing epitope is capable of selecting for viral escape variants during the acute or periacute phase of HIV infection is of high avidity.

### CROSS-REFERENCE TO RELATED APPLICATION

This application claims priority from U.S. Ser. No. 60/169,412, filed Apr. 12, 2000. Ser. No. 60/169,412 is incorporated by reference herein.

### STATEMENT REGARDING FEDERALLY SPONSORED RESEARCH OR DEVELOPMENT

### BACKGROUND OF THE INVENTION

Developing an effective vaccine for HIV would prevent considerable suffering, particularly in Africa where over 30 million individuals are infected (Working Group on Global HIV/AIDS and STD Surveillance, U. W., 1998). While many vaccine regimens have demonstrated the ability to contain viral infections in macaques challenged with SIV or SHIV, these vaccines have yielded equivocal results (Hanke, T., et al., J. Virol. 73:7524-7532,1999; Daniel, M. D., et al., Science 258:1938-1941, 2000; Desrosiers, R. C., et al., Proc. Natl. Acad. Sci. USA 86:6353,1989; Hirsch, V. M., et al., J. Infect. Dis. 170:51-59, 1994; Mossman, S. P., et al., J. Virol. 70:1953-1960, 1996; Murphy-Corb, M., et al., Science 246,1293, 1989; Robinson, H. L., et al., Nat. Med. 5:526,1999; Barouch, D. H., et al., Science 290:486-492, 2000). After more than a decade of intense vaccine research, traditional approaches to vaccine design for HIV have still not definitively identified the correlates of a protective immune response. Most primary HIV strains are resistant to neutralization by antibody, so it is unlikely that these responses would be broadly effective against field strains of HIV (Moore, J. P., et al., J. Virol. 66:235-243, 1992; Moore, J. P and Ho. D. D., Aids 9:S117-S136, 1995). Unlike neutralizing antibodies, which are all directed against surface-exposed regions of the viral envelope, the specificity of cellular immune responses are largely determined by primary amino acid sequences. Therefore, each viral protein contains potential targets for cellular immune responses. It is not currently known whether any regions of the virus are particularly immunogenic as a consequence of their innate biological function. Thus, identifying the most immunogenic cellular immune responses and incorporating these responses into a vaccine might be one way of reducing viral replication and shifting the balance in favor of the host.

During the first weeks of HIV and SIV infection, the virus replicates at high titer. After a peak in viremia approximately three weeks post-infection, the amount of circulating virus declines dramatically. By twelve to sixteen weeks of infection the titer of virus in the plasma stabilizes at a level that is commonly known as the viral set point. This viral set point is inversely correlated with length of survival post-infection (Mellors, J. W., et al., Ann. Intern. Med. 122:573-579,1995; Mellors, J. W., et al., Science 272:1167-1170, 1996; Mellors, J. W., et al., Ann. Intern. Med. 126:946-954, 1997). If an individual's viral load remains high after the acute phase, then it is probable that the patient will succumb to AIDS rapidly in the absence of anti-retroviral therapy. While a patient's viral set point is likely the product of multiple factors, the strength of the patient's cellular immune response likely plays an important role in determining the set point.

Several studies have revealed that cytotoxic T lymphocytes (CTL) are largely responsible for control of the initial viral replication. First, it has been observed that neutralizing antibodies do not develop until later in infection (Legrand, E., et al., AIDS Res. Hum. Retroviruses 13:1383-1394,1997; D'Souza, M. P. and Mathieson, B. J., AIDS Res. Hum. Retroviruses 12:1-9, 1996). Second, CTL responses emerge coincident with declining acute phase viral RNA levels (Borrow, P., et al., J. Virol. 68:6103-6110,1994, Koup, R. A., et al., J. Virol. 68:4650-4655, 1994; Yasutomi, Y., et al., J. Virol. 67:1707-1711,1993). Finally, SIV-infected macaques depleted of CD8 positive lymphocytes do not effectively resolve their acute infection, implicating antigen-specific CTL in control of viral replication (Metzner, K. J., et al., J. Exp. Med. 191:1921-1932, 2000; Hin, X., et al., J. Exp. Med. 189:991-998, 1999; Schmitz, J. E., et al., Science 283:857-860, 1999; Matano, T., et al., J. Virol. 72:164-169, 1998). Unfortunately, while those of skill in the art now possess a greater understanding of the cellular CTL responses directed against HIV and SIV, we still do not understand which of the many CTL responses are best capable of controlling HIV, and therefore should be engendered by a vaccine.

### BRIEF SUMMARY OF THE INVENTION

In one embodiment, the present invention is a method of identifying at least one CTL-inducing epitope from at least one HIV protein, wherein the immune response directed against this epitope is capable of selecting for viral escape variants during the acute or periacute phase of infection, wherein the method comprises the steps of (a) examining a nucleic acid sequence encoding at least one HIV protein from at least one HIV-infected patient, wherein the sequence encoding the expressed protein is examined in the first six months after infection, to identify at least one region of the HIV protein that is variable as compared to the sequence of the protein at an earlier time point in infection, wherein the variable region indicates a CTL-inducing epitope, and (b) confirming that an immune response directed against the CTL-inducing epitope is capable of selecting for viral escape variants during the acute or periacute phase of HIV infection.

Preferably, the protein is examined through sequencing of the virus from an individual between 0 and 24 weeks after infection and is selected from the group consisting of Gag, Env, Pol, Rev, Nef, Tat, Vpx, Vpu, and Vif.

The method preferably comprises the step of testing peripheral blood mononuclear cells (PBMC) from HIV infected patients in the first six months after infection to confirm that CTL responses to the CTL-inducing epitope of step (a) are of high avidity.

In a most preferred embodiment, a minimal peptide needed to elicit the CTL response is determined.

In another embodiment, the invention is a method of identifying at least one CTL-inducing epitope from an RNA virus, wherein an immune response directed against the epitope is capable of selecting for viral escape variants during the acute or periacute phase of viral infection, wherein the method comprises the steps of (a) examining the viral nucleic acid sequences from virus-infected patients in the first six months after infection to identify at least one region of the virus that is variable, wherein the variable regions indicate a CTL-inducing epitope and (b) confirming that the epitope is capable of selecting for viral escape variants during the acute or periacute phase of viral infection.

In another embodiment, the present invention is a vaccine comprising a nucleic acid encoding at least one CTL-inducing epitope selected by the methods described above.

It is an object of the present invention to provide epitopes suitable for an HIV vaccine.

It is another object of the present invention to provide epitopes for any RNA virus vaccine.

Other objects, features and advantages of the present invention will become apparent after observation of the claims, specification and drawings.

### DETAILED DESCRIPTION OF THE SEVERAL VIEWS OF THE DRAWINGS

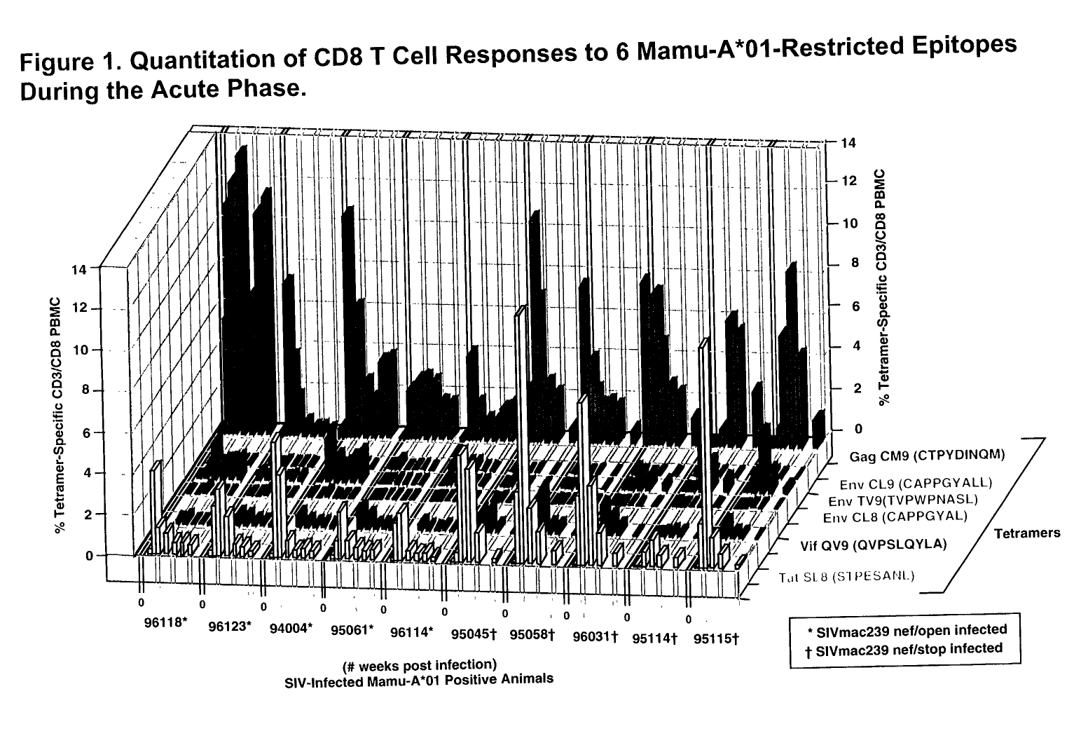

FIG. 1 is a bar graph describing quantitation of CD8-positive T-lymphocyte responses to various Mamu-A*01-bound peptides.

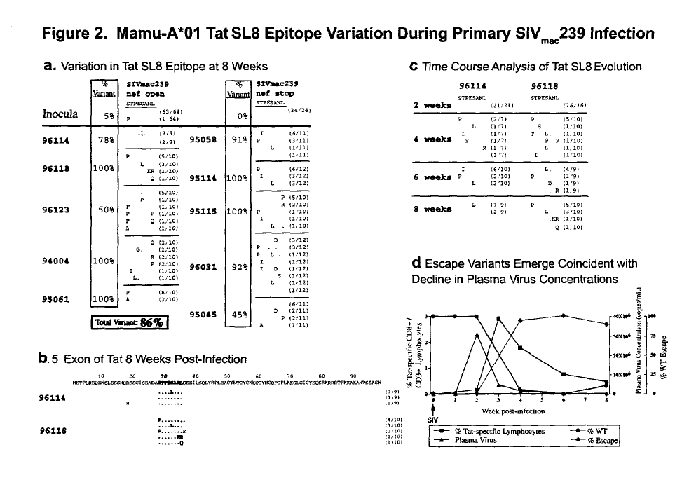

FIG. 2 is a sequence comparison describing variations present in the SL8 epitope of all 10 Mamu-A*01 positive animals after acute SIV infection.

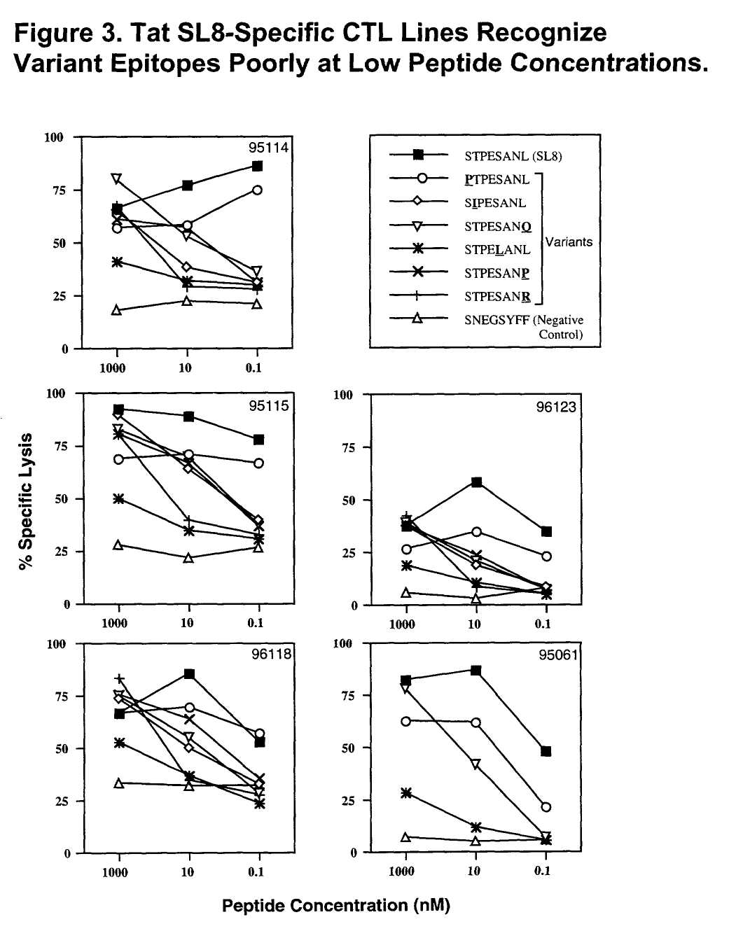

FIG. 3 is a set of six graphs describing CTL analyses of CD8-positive T-lymphocytes stimulated with the SL8 peptide.

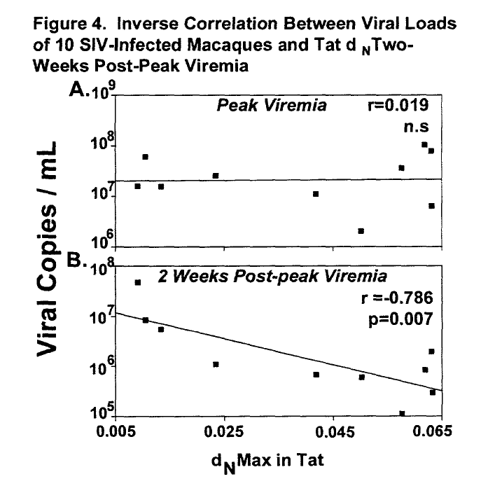

FIGS. 4A and B describe inverse correlation between plasma viral concentration and peak d_{N} in Tat.

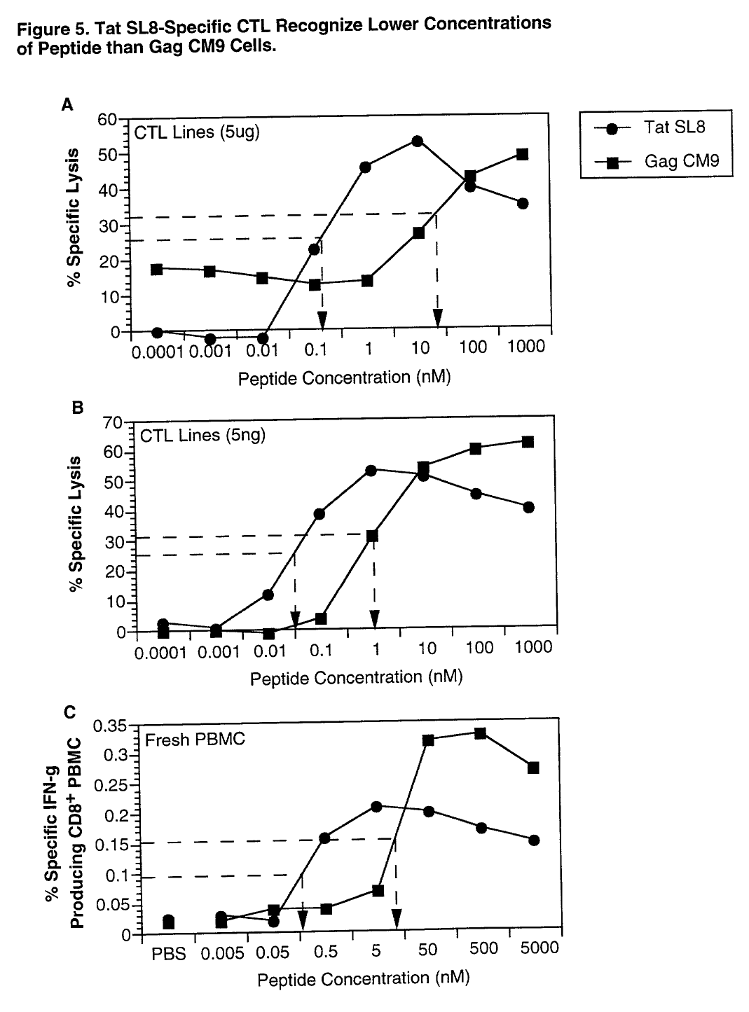

FIGS. 5A, B and C are graphs demonstrating that Tat SL8-specific CTL recognize lower concentrations of peptide than Gag CM9 cells. FIG. 5A is a graph of % specific lysis versus peptide concentration for CTL lines generated with 5 μg of peptide. FIG. 5B is a graph of % lysis versus peptide concentration for CTL lines generated with 5 ng of peptide. FIG. 5C is a graph of % specific INF-γ producing CD8⁺ PBMC versus peptide concentration.

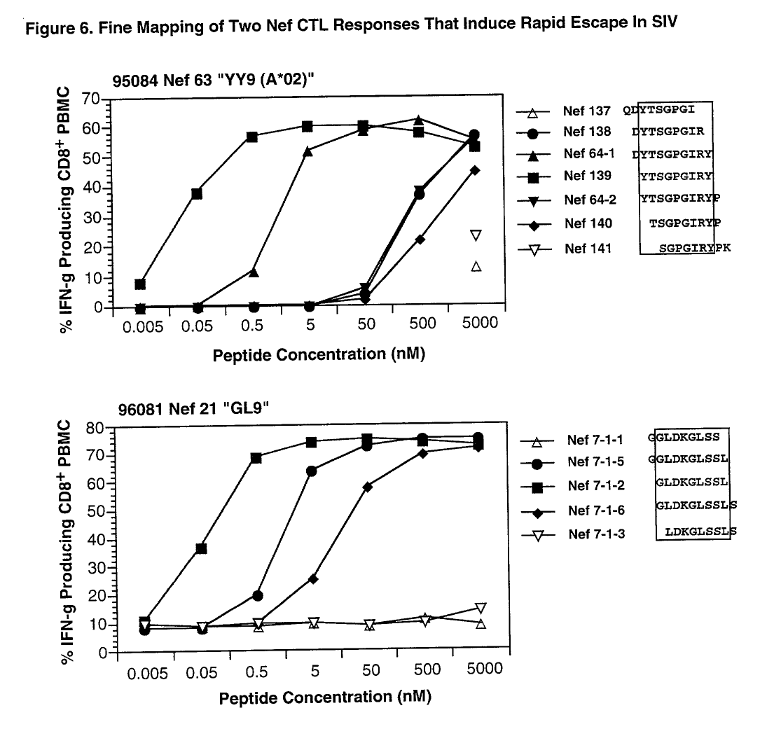

FIGS. 6A and B are a set of graphs demonstrating that fine mapping of additional SIV CTL responses induce rapid escape. FIG. 6A fine mapping of the 95084 Nef63YY9 epitope. FIG. 6B fine mapping of the 96081 Nef21 BL9 epitope.

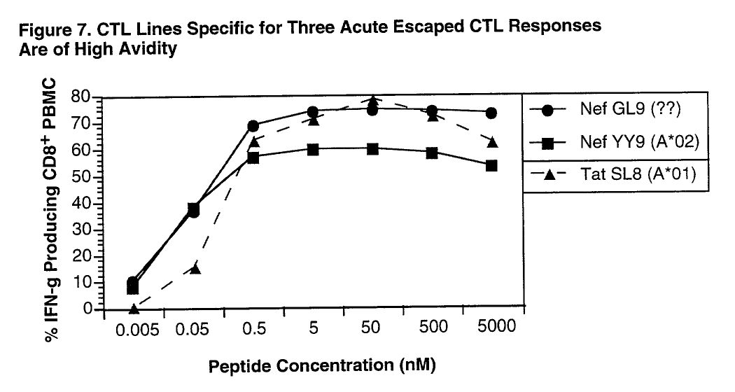

FIG. 7 is a graph of % IFN-γ producing CD8⁺ PBMC versus peptide concentration and demonstrates that CTL lines specific for additional acute escaped CTL responses are of high avidity.

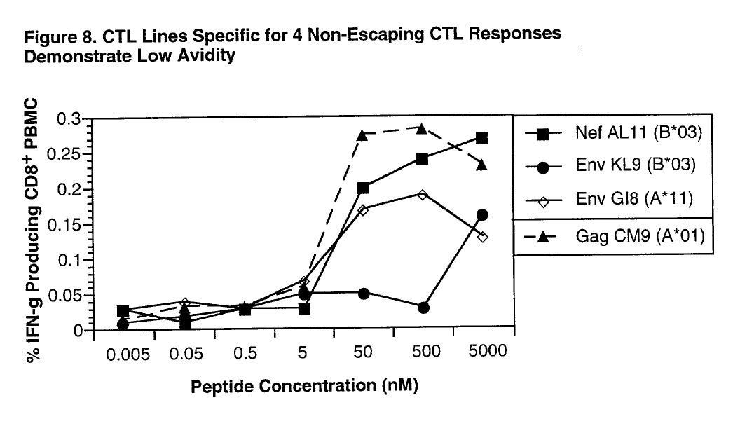

FIG. 8 is a graph of % IFN-γ producing CD8⁺ PBMC versus peptide concentration and demonstrates that PBMC specific for additional non-escaping CTL responses demonstrate low avidity.

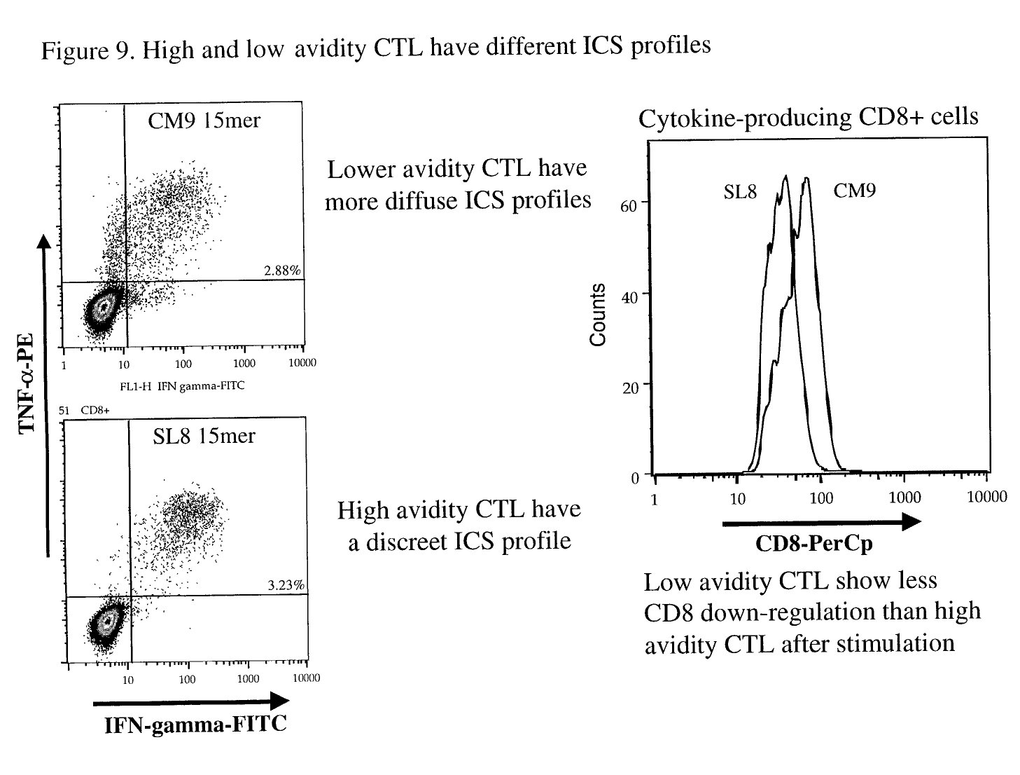

FIG. 9 is a set of graphs demonstrating that high and low avidity CTL responses have different ICS (intracellular cytokine staining) profiles and that high avidity CTL demonstrate NCD8 down regulation.

### DESCRIPTION OF THE INVENTION

A. In General

Induction of strong cellular immune responses against various proteins of SIV by the majority of vaccines has failed to significantly reduce the initial peak of viremia. Some regimens, however, have reduced set point after challenge against certain strains of SIV. In most of these studies we believe that, given the massive replication which occurred during the acute phase, it is likely that a sufficient number of mutant viruses were spawned early after infection which eventually resulted in the escape of the virus from many host immune responses. Therefore, our approach is to try to reduce initial virus peak so that we can prevent the spawning of new variants that will eventually escape the host's immune responses.

In order to accomplish this we believe that an effective vaccine will have to induce those immune responses that are most capable of exerting pressure on the virus. The majority of vaccines attempt to induce CTL immune responses against the whole virus. A potentially more effective approach, and the approach of the present invention, is to select only a few (1-4) short regions of the virus to direct CTL immune response towards such that these CTL responses are stronger and not diluted out. However, no one has ever defined which of the many HIV-specific CTL immune response might be best able to control HIV. Our approach details methods capable of identifying the best CTL responses.

While it has previously been shown that virus-specific CTL exert selective pressure on SIV in vivo during the chronic phase of HIV and SIV infection, these data are still a matter of some debate. The difficulty in finding unequivocal evidence for escape in HIV-infection may be related to a variety of factors. Patients are infected with heterogenous inocula that is often inaccessible to the investigator. Furthermore, most escape studies are carried out during the chronic phase of HIV infection because patients rarely know when they had been infected. Given that the peak of the antigen-specific CD8 response to HIV and SIV occurs within the first few weeks of infection, it is possible that the majority of escape occurs during this acute phase. Indeed two cases of escape at or around the time of seroconversion have been reported in HIV-infected individuals. However, these two case reports remain as isolated examples of viral evolution immediately after the acute phase.

The present invention discloses a different approach to vaccine design for HIV. In brief, we use two independent but supporting approaches to analyze (I) viral evolution and (II) high avidity CTL responses in HIV infected patients. These two independent approaches are both capable of identifying unique CTL responses against HIV and may be used separately or in combination. These unique responses are distinguishable from the majority of CTL responses detected in HIV infected patients and should represent those CTL responses best capable of controlling HIV. We reason that discovery of regions of the virus that vary, or CTL responses that are of high avidity, will allow us to design a rational, novel vaccine approach to HIV. Example 1 (Escape) and Example 2 (Avidity) describe our recent work with this approach and should be examined to understand the present invention.

B. A Method for Making an HIV Vaccine

Attempts to develop a safe and effective vaccine for HIV using traditional approaches such as live-attenutated, heat-killed, or recombinant (protein) approaches have all failed. We will, therefore, take a radically different approach to HIV vaccine design by (I) analyzing viral evolution (escape) and (II) analyzing for high avidity CTL responses within the first 24 weeks after HIV infection in humans. We predict that these methods will identify unique CTL responses with a superior ability to control HIV, which could then induce these CTL responses. It is likely that traditional approaches to HIV vaccine design will induce broad CTL responses that will not focus the cellular immune response against the most important CTL epitopes. This likely will have the effect of diluting out many of the protective CTL responses.

While our two approaches can independently assess for unique CTL responses with a superior potential to control HIV, they are also complementary. Our research suggest that these two biological phenomenons, the ability of a particular CTL response to induce viral escape during the acute phase of SIV infection and the avidity of the CTL response, are inherently linked. This approach will allow us to define those regions of the virus against which the strongest immune responses, which are not tolerated by the rapidly mutating HIV, are directed. We envision that potent vaccine-induced cytotoxic T-lymphocyte (CTL) responses against these defined regions of the virus will be capable of blunting infection in individuals infected with HIV.

Results from clinical trials and animals studies indicate that it is unlikely that vaccine-generated antibody responses will generate sterilizing immunity. In contrast, strong cellular immune responses or CTL (cytotoxic T lymphocytes), both natural and vaccine-induced, are showing promise for their potential to control HIV and SIV (simian immunodeficiency virus) infections. Furthermore, there is data to suggest that strong vaccine-induced antibodies may compromise the generation of the potentially more important cellular immune responses.

The shortcomings of using traditional approaches to design an effective vaccine against HIV are likely due to the inability of these vaccines to direct immune responses against the proper regions of the virus. With this in mind, the concept of epitope-based vaccines has recently emerged. This approach strings together short specifically-selected regions of the virus (epitopes) for cellular immune responses to be directed against. Epitope-based vaccines have shown promise against SIV and other pathogens by selecting those epitopes to which immune responses have been observed to be generated against during natural infections. However, despite a growing knowledge of HIV- and SIV-specific CTL epitopes, as of yet there has been no clear indication of which particular epitopes may be better able to protect against HIV and should be inserted into epitope-based vaccines.

The majority of regions (epitopes) from HIV against which HIV-specific cellular immune responses have been described have been identified from chronic-stage (>1 year post-infection) HIV-infected individuals. These epitopes recognized during the late stages of infection, however, likely represent secondary cellular immune responses which have arisen only after the primary cellular immune responses, which are active during the acute stage (2-8 weeks post-infection), have lost their ability to kill virally-infected cells. This inability to recognize acute-stage virus is due to the accumulation of mutations which likely develop within the virus during the first few weeks after infection.

B.1 Escape

There is extensive evidence indicating that some of these “escape” mutations occur in those regions of HIV to which particular cellular immune responses are uniquely capable of controlling viral replication. This escape permits certain variants (escapees) of the virus to continue to replicate unchecked. While some of the secondary cellular immune responses are capable of controlling viral replication to some degree, as evidenced by the eventual accumulation of escape mutations in certain regions (epitopes) of the virus late in infection, unfortunately many of these late immune responses are not particularly effective at controlling viral replication. Many more do not even accumulate mutations. We concluded, therefore, that these latter-mentioned late immune responses that are currently being used in clinical HIV vaccine trials do not represent particularly good candidate epitopes for inclusion in an HIV vaccine.

We have data suggesting that virus isolated during the acute phase (i.e. at 4 and 8 weeks post-infection) of SIV-infected rhesus macaques has “escaped” from a potent CTL response. This is the first evidence of such a rapid and extensive escape of the virus from a strong CTL response during the acute phase. Two weeks following infection with HIV or SIV there is a peak of viral replication. Virus replication then declines to a more stable “set point” following engagement of the CTL responses. At peak viremia (2 weeks post-infection), only wild-type virus was found to be replicating. However, at 4 and 8 weeks post-infection the majority of wild-type virus was absent from these SIV-infected animals, and was replaced by an escaped virus which had accumulated extensive mutations in the region of the virus to which an early potent CTL response had been directed. This strongly suggests that the CTL responsible for selecting the escape variants had killed all cells actively producing the wild-type virus. In addition, these findings suggest that the virus replicating at the set point are largely comprised of “escaped” viruses. We have now identified these early potent CTL responses capable of inducing viral mutation in various proteins of SIV, including Tat and Nef.

This data strongly suggests that immune responses directed against specific regions of the virus (epitopes) are responsible during the acute phase for controlling viral replication. The accumulation of mutations within these epitopes is one of the two methods we have now identified which defines them as particular potent vaccine candidate epitopes.

To identify the highly effective responses during early infection, we would first establish a cohort of high-risk, uninfected individuals. These individuals could include intravenous drug users (IVDUs), children born to HIV-infected mothers, or individuals living within communities with a high frequency of HIV infection where infection rates would presumably be relatively high.

In each of these individuals, we would prospectively obtain frequent (monthly) blood draws. To determine whether these individuals become HIV positive, lymphocytes and plasma would be purified from the whole blood using Ficoll gradient centrifugation. HIV viral RNA, if present, would be isolated using commercially available viral RNA purification kits.

The viral RNA samples would be reverse transcribed and subjected to polymerase chain reaction (RT-PCR) in the presence of HIV-specific oligonucleotides. A positive RT-PCR result indicates the presence of HIV vRNA in the blood of the individual. Generally, HIV vRNA can be detected in plasma within 7-10 days of infection.

Upon a positive HIV test result, an individual will be asked to submit to weekly blood draws throughout the duration of acute infection. This will allow us to isolate and sequence the earliest possible viral RNA from a patient. This early sequence, which as we have demonstrated in rhesus monkeys does not begin to escape at 2 weeks post-infection, should not have any new mutations. Therefore, this very early sequence can serve as an ‘index sequence’ template to allow us to identify an new acute viral escapes. In any patient the earliest possible viral sequence obtained will have to represent the ‘index sequence’ for that patient against which all future sequences from that individual will be compared.

Once an individual tests positive for HIV, we will begin to identify potent CTL responses. First, we will design a series of oligonucleotides that will allow us to amplify the entire 9.5 Kb HIV genome. The amplicons obtained by amplification with these primers will overlap each other by at least 100 base pairs and will be approximately 1.0-2.5 Kb in size. The vRNA samples derived from the first positive test sample will be subjected to RT-PCR using each of the primer pairs needed to amplify the entire genome. The cDNA sequence of each PCR amplicon will be determined by sequencing both strands of the cDNA. This initial screen will identify the index sequence that approximates the infecting HIV strain. The same procedure of HIV genome amplification and sequencing will then be repeated on samples derived at weekly intervals throughout acute infection. Sites in the resultant sequence where two or more nucleotides coexist at a single position in the virus indicate a mixed-population in the sample. These sites of mixed-base heterogeneity may indicate positions where variant virus is competing with wild-type virus (index sequence) under selective pressure from CTL responses. By comparing the contemporary sequence to the index sequence, we will identify sites that may be evolving under selective pressure from the CTL response.

To determine whether these sites of active viral evolution during early infection correspond to epitopes recognized by highly effective CTL responses, we will assay lympocytes cyropreserved from the infected individual during early infection for recognition of this viral sequence. Synthetic peptides approximately 15 amino acids long corresponding to the sequence of the infecting virus will be made. These peptides will be used in conventional assays for cellular lymphocyte reactivity using effector cells from the infected individual. If these peptides are immunogenic, it is likely that these peptides encompass a CTL epitope that is evolving under selective pressure against the virus. The minimal, optimal peptide needed to elicit the CTL response can then be determined using truncated and overlapping synthetic peptides.

B.2 Avidity

We are accumulating data to suggest that within the mileau of CTL responses to a given pathogen some CTL responses are much more effective than others in controlling viral infections. During a CTL response many different regions (epitopes) of a virus are recognized by an equal number of different CTL responses. Reports suggest that some of the CTL responses recognizing particular epitopes are superior in their ability to rapidly and effectively kill a virally infected cell than CTL responses directed against other epitopes. The distinction between these different sets of CTL has been attributed to the avidity of the CTL response recognizing a particular epitope. Avidity defines the biochemical interaction between the CTL and the virally infected cell, with some CTL/infected cell complexes interacting very strongly, while other CTL/infected cell complexes interacting only weakly. Avidity can be easily determined by the level of a particular antigen (epitope) on the surface of a virally infected cell that is required to trigger lysis of the cell by a particular CTL. Some CTL responses, termed “high avidity CTL” are capable of recognizing virally infected cells with extremely low levels of epitope on the surface. This is in contrast to the majority of CTL responses that require a higher threshold of epitope to recognize infected cells. This difference in avidity can be very important during a viral infection since high avidity CTL may be better able to recognize a recently infected cell producing only low levels of epitope on the surface. Rapid destruction of a recently infected cell, before production of newly formed infectious virus, would effectvely prevent dissemination of the virus. In contrast, low avidity CTL may only be effective at recognizing a cell in more advanced stages of infection when there is a larger amount of antigen on the surface.

The importance of these high avidity CTL has been demonstrated very eloquently in adoptive transfer experiments in mice against both lymphocytic choriomenengitis virus (LCMV) and vaccinia virus. These studies demonstrated an immense advantage of high avidity CTL over low avidity CTL to control infections. Similarly, high avidity CTL against self-antigens have demonstrated superior antitumor efficacy in vivo in mice. However, despite this understanding of the greater potential of high avidity CTL to protect against viral infections, no data exists for such CTL responses in HIV.

We now have data (see Example 2) that the CTL responses we have identified as being capable of rapidly inducing escape in SIV, in fact, represent high avidity CTL. To determine this we examined the avidity of some of the various SIV-specific CTL responses we had identified to which definitive epitopes had been defined. This required testing the ability of peripheral blood mononuclear cells (PBMC) from SIV infected rhesus macaques to recognize cells pulsed with varying concentrations of these different CTL epitopes. We separated our epitopes into two distinct groups, those epitopes known to mutate rapidly during the acute phase of SIV infection and those that failed to mutate rapidly. High avidity CTL would be capable of recognizing very low concentrations of a particular peptide while low avidity CTL would require 10-100 fold more peptide. We had previously identified three CTL epitopes, in the SIV proteins Tat and Nef, which had rapidly escaped after SIV infection. We observed that these CTL responses were, in fact, capable of recognizing extremely low levels of peptide, and were deemed high avidity CTL. In contrast, those epitopes that did not mutate early only recognized very high concentrations of peptide.

Our findings, therefore, represent the first data linking high avidity CTL, which are known to be superior in protecting against viral infections, with CTL responses capable of inducing escape. This data strongly suggests that those epitopes that are high avidity can potentially control an HIV or SIV infection by exerting selective pressure on the virus. This selective pressure is evidenced by viral escape in a rapidly mutating virus such as HIV. The accumulation of mutations within these epitopes and the high avidity of the CTL responsible for inducing these mutations uniquely defines these CTL epitopes as particular potent vaccine candidate epitopes for HIV.

One caveat, however, is that once an epitope mutates, the frequency of cells recognizing the original epitope significantly declines. Therefore, identifying high avidity CTL responses against HIV by testing PBMC against various peptide concentrations will likely prove difficult. This could be overcome by generating in vitro CTL lines against the region of the virus to which CTL escape has been observed. Alternatively, the only time during which these cells would be of sufficient frequency to test without the use of CTL lines would be during the acute phase. Proper enrollment of uninfected, high-risk individuals may allow for direct testing of acute phase PBMC for high avidity CTL.

We have questioned why high avidity CTL in HIV and SIV infections has not been previously observed. Few of the viruses studied extensively to date mutate as quickly and error-prone as HIV and SIV. This ability to mutate, we believe, has in essence masked the ability to detect high avidity CTL to HIV and SIV. In many viral infections distinguishing high avidity CTL responses from low avidity CTL responses would be relatively easy because simple peptide titrations could be performed for each epitope. However, in the setting of an RNA virus, such as HIV or SIV that has the capacity to mutate very rapidly these regions of the virus to which the CTL responses are directed are the first regions of the virus to mutate. Once an epitope has escaped the frequency of these T-cells declines rapidly making identification of these responses very difficult.

Therefore, in the setting of an HIV or SIV infection these high avidity and rapidly escaping CTL responses may be extremely difficult to identify using traditional approaches. In our case, it was only through sequencing of the SIV virus in the acute phase that these acutely escaping epitopes were identified. Once the region of the virus to which these unique CTL responses were known we were able to more sensitively measure the avidity of these CTL responses and confirm that they represented high avidity CTL with the likely potential to represent highly protective CTL responses. Therefore, it is likely that given HIV and SIV's propensity to escape from these high avidity CTL, thereby reducing the levels of these CTL, that high avidity CTL responses in HIV and SIV have not been previously identified.

However, while the peak CTL responses are not attainable, plasma and PBMC samples from the peri-acute phase are often available. In these and later chronic phase samples the virus will still maintain the CTL mutations induced by these high avidity CTL making identification of the high avidity CTL epitopes possible. Similarly, low level memory cells will be present in peri-acute PBMC samples allowing for generation of CTL lines.

High avidity CTL responses can also be identified in uninfected individuals who have been vaccinated against HIV or SIV. The ICS profiles for interferon-gamma (IFN-g) of low and high affinity CTL are different. High avidity CTL stain much brighter with anti-cytokine antibodies such as IFN-γ and tumor necrosis factor-alpha (TNF-α) after stimulation with the same peptide concentration (FIG. 9, Example 2) compared with low avidity CTL. High avidity CTL also possess a greater degree of CD8 down-regulation in comparison to low avidity CTL after stimulation with their cognate ligand (peptide) at the same concentration (FIG. 9, Example 2). These staining profiles are seen both in infected animals and in SIV-vaccinated animals. Therefore, in the setting of HIV we could potentially use this additional approach to define high avidity CTL responses in individuals currently enrolled in various HIV vaccine clinical trials.

Our data examining CTL escape combined with our new data linking high avidity CTL to these escaped epitopes suggests that traditional approaches to identifying these high avidity CTL responses in HIV may be labor intensive. This is because once the epitopes mutate the frequency of these high avidity CTL drop dramatically, making their detection through cellular assays extremely difficult. Sequencing of the virus, on the other hand, is independent of the level of these CTL and therefore is not similarly affected. When combined, however, these two approaches allow for rapid identification of high avidity CTL responses which possess a unique ability to control HIV.

Alternately, we can directly identify which, among the database of already identified HIV epitopes, are high avidity. First, we would obtain class-I defined target cells to be used in these assays. We would then pulse these target cells with varying amounts of synthetic peptide corresponding to the previously identified epitopes. Lymphocytes from early HIV infection will be used as effectors in these assays. We will identify lymphocyte specificities that are activated under conditions of low peptide concentration, and then analyze the viral sequence of these epitopes. Similarly, we can assess for high avidity T-cell responses by examining the IFN-γ ICS profile of thawed PBMC in response to a particular peptide. High avidity CTL should be much brighter for anti-IFN-γ antibody staining and also exhibit a greater degree of CD8 downregulation compared to low avidity CTL.

B.3 Combination of the Two Approaches

The present invention involves a radically different approach to HIV vaccine design: instead of determining to which regions of the virus are broad immune responses being generated, we will ask the virus which specific immune responses it cannot tolerate and focus vaccine-induced immune responses against these regions. To do this, we will analyze viral evolution (escape) in humans during the first year after HIV infection, particularly during the acute phase. Variable regions of the virus will indicate selective pressure by the host on the virus and the subsequent escape of the virus from these potent immune responses. Therefore, vaccination to induce these potent cytotoxic T lymphocyte (CTL) responses will be better able to blunt infection in individuals infected with HIV. The present invention differs from current approaches which have either targeted the entire virus, or directed the immune response to any and all epitopes which have been defined. Many of these epitopes are not critical for controlling viral replication—as evidenced by the lack of accumulation of mutations in these regions of the virus even during the chronic stage. Therefore, many vaccine regimens currently being tested are likely not focusing immune responses against the critical regions of HIV.

Our hypothesis implies that if we vaccinate with particular regions of the virus which induce CTL capable of selecting these new variants, we may be able to reduce initial virus loads. We will, therefore, use these “escaped” regions of the virus to induce robust CTL. We predict that these vaccine-induced CTL responses should reduce the initial peak of viremia such that spawning of new escape variants should be prevented and the immune system will gain the upper hand.

B.4 Specific Embodiments

i. Vaccine

A vaccine for HIV of the present invention needs to induce strong CTL responses against particular regions or epitopes of HIV. Thus, protein vaccinations are not likely to induce sufficiently strong CTL responses against the important CTL epitopes to reduce the initial viremia sufficiently to prevent progression to disease. This is because whole protein vaccines will induce very broad but low level CTL responses to many different regions of HIV as opposed to the most important regions.

One preferred embodiment of the vaccine of the present invention is a DNA prime, MVA (modified vaccinia Ankara) vaccine using a string of CTL and HTL epitopes (identified as described below) in the vaccination of humans. Preferably, one would use only epitopes since this would focus immune responses to preferred regions. This will likely induce strong CTL which should reduce initial viremia and prevent the spawning of new mutant viruses. The immune system of the host will be favored by this reduction in viremia, preventing the generation of escape mutants.

A typical DNA vector construct for the DNA vaccinations would include a generic DNA plasmid backbone, a CMV (cytomegalovirus) enhancer/promoter, CMV enhancer/immediate early promoter/intron A region, and a polyepitope (string of epitopes) as previously defined. The MVA would contain this same insert ligated directly into a shuttle vector for transformation of wild-type MVA. Preferably, the polyepitope would comprise acutely escaping or high avidity CTL epitopes described above.

ii. Identification of CTL-Inducing Epitopes

One will need to identify regions of SIV or HIV that would likely be part of an epitope-based vaccine.

Our data suggest that one should preferably sequence full-length HIV from HIV-infected patients because variation will be present in CTL epitopes early on after infection as escape will have occurred. Thus, as described above, a preferred strategy would be to sequence the HIV nucleic acid from recently infected individuals, preferably within 24 weeks of infection, defining the variability and then making synthetic peptides to that region. Defining the variability would require sampling of viral RNA from an infected individual at various time points after infection. Sites in the resultant sequence where two or more nucleotides coexist at a single position in the virus indicate a mixed-population in the sample. These sites of mixed-base heterogeneity may indicate positions where variant virus is competing with wild-type virus under selective pressure from CTL responses. An index sequence for each individual, defined as the earliest viral RNA sample derived from that patient, will serve as a reference sequence. This index sequence, if possible, should be derived from plasma isolated during peak viremia at a time when viral mutations should be at a minimum. By comparing the contemporary sequence to the index sequence, we will identify sites that may be evolving under selective pressure from the CTL response.

One would preferably use HIV molecules from 20 individuals, more preferably 50 individuals and most preferably 100 individuals to define variability. The larger the sample size the better indication of the nature of acute escape and viral variability and the greater potential to identify a significant number of escaped epitopes.

One would then need to confirm that a cellular immune response is in fact responsible for the mutation, that is, capable of selecting for viral escape variants during the acute (2-8 weeks post infection) or periacute phase (within 16 weeks post infection) of viral infection. This is necessary because some mutations can spontaneously arise or arise to improve the general fitness of the virus. The immune response responsible for the mutation can be confirmed by generating in vitro CTL lines. These lines are generated using PBMC from the patient displaying the viral mutation and stimulating these PBMC with autologous B lymphoblastoid cell lines (BLCL) and a peptide of 15-20 amino acids in length corresponding to the region of the virus with the mutation. This will allow one to generate CTL lines to the region of HIV where the escape is detected. These CTL lines can then be used to map the epitope using overlapping peptides. Simultaneous MHC typing of the host and testing for reactivity to this epitope against a panel of MHC-typed BLCLs in ICS assays will allow one to also define the restricting MHC class allele. One can, therefore, define CTL epitopes in any HIV protein.

As a basis for comparing individual T-cell responses we will measure the concentration of peptide required to induce 50% of the maximal response (½ₘₐₓᵢₘₐₗ) observed for that particular CTL line or PBMC sample. That is, at very high peptide concentrations a maximal response will be elicited and measured as either % specific lysis for 51Cr-release CTL assays, or % IFN-γ production for ICS assays. Therefore, the concentration of peptide required to induce 50% of that maximal response will be termed the ½ₘₐₓᵢₘₐₗ response for that particular peptide. Generally we observe that high avidity CTL demonstrate a ½ₘₐₓᵢₘₐₗ response at peptide concentrations of 0.5 nM or less while low avidity CTL demonstrate a ½ₘₐₓᵢₘₐₗ response at peptide concentrations of 5.0 nM or higher when tested using intracellular cytokine staining (ICS) methods or ⁵¹Cr-release assays.

Once CTL lines for these epitopes are generated, one can define the avidity of these CTL responses through the testing of peptide dilutions of each epitope in intracellular staining experiments or 51Cr-release CTL assays (as described below). This generally leaves at least a 1-2 log range of peptide concentrations to distinguish between high and low avidity CTL responses.

Therefore, CTL responses which are unique compared to the majority of CTL responses can be identified through one of two methods. The first method involves sequencing of virus from an HIV-infected patient to identify regions of the virus which have escaped early after infection. The exact epitope(s) can then be confirmed through cellular assays. The second method involves testing the avidity of a particular CTL response through peptide dilutions with CTL lines or whole PBMC using 51Cr-release assays or ICS assays.

iii. Other RNA Viruses

One could then apply these above-mentioned strategies for unique CTL epitope identification to any other RNA virus because of the propensity of these viruses to escape. This is because for escape to occur two basic criteria must be satisfied which are properties of RNA viruses. First, a large pool of viral variants, each of which could potentially represent an “escape” mutant need to exist which are most likely generated from erroneous viral replication. RNA viruses, unlike many other viruses, all require either an RNA-dependent RNA polymerase or an RNA-dependent DNA polymerase which by nature accumulate substantial errors during each round of viral replication.

A second criteria for CTL escape is viral tolerance for variation. All viruses have functionally and structurally important domains, although the plasticity of these domains can vary. The pool of viruses in an individual, therefore, are likely restricted to variants that can be tolerated by the virus. Viruses with diverse genetic subtypes, indicating a plurality of mechanisms capable of performing necessary functions, are more likely to accommodate variation than viruses with more uniform genotypes. As RNA viruses are more diverse than DNA viruses, they should be more able to accommodate CTL escape variants.

Examples of suitable RNA viruses include Influenza, Hepatitis, Polio virus, Yellow Fever, and Dengue virus. The methods and the principles behind the method are directly adaptable and would simply require identification of acutely infected individuals, isolation of virus and PBMC samples from these patients, and viral sequencing, epitope mapping and avidity determination.

iv. Vaccination Strategies

One embodiment of this invention is a commensal vaginal bacterium that expresses the acutely escaping or high avidity CTL epitopes in the vaginal mucosa. This would stimulate CTL at the relevant site of challenge.

This bacterium could be introduced to the mucosal surfaces as part of a vaccination regimen or as part of a spermicidal contraceptive.

Another strategy is a simple DNA vaccination to the skin (×3) using vectors expressing these CTL epitopes with or without a subsequent MVA (×1) boost expressing the epitopes. Helper T lymphocyte (HTL) epitopes may also be included to induce CD4 T-cell help. An alternative approach would be to use a simple DNA vaccination to the skin (×3) expressing these epitopes followed by subsequent adenovirus (×1) boost expressing these epitopes

v. Identification of HTL Epitopes

HTL (helper T lymphocytes) are similar to CTL in that they represent another type of immune response important in HIV infections that may also be identified through this method. The methods and vaccines above can be useful to identify and use HTL-inducing epitopes. HTL epitopes would be defined using the exact methods outlined above except that rather than looking for CD8⁺ CTL responses one would examine CD4⁺ T-cells for HTL responses using ICS as a measure of IFN-g production. 51Cr-release assays would not generally be applicable to HTL responses.

C. Supporting Data

C.1 Escapes

HIV and SIV infections are characterized by early peaks of viral replication that decline coincident with the development of strong cytotoxic T lymphocyte (CTL) responses. Our data show that Tat-specific CTL select for new viral variants during the acute phase of infection. At eight weeks post-infection, the majority of the replicating virus has escaped recognition by these CTL. This implies that wild-type virus replication has been controlled by Tat-specific CTL. We envision that induction of CTL against viral proteins expressed early during the viral life cycle is important in formulating an effective HIV vaccine.

i. CD8 Positive Lymphocytes Recognize an Immunodominant Epitope in Tat

To determine whether the initial strong CTL response can select for escape variants, we followed CD8 positive lymphocyte responses to 6 different Mamu-A*01-restricted epitopes in ten Mamu-A*01 positive macaques infected with molecularly cloned SIVmac239. Surprisingly, all Mamu-A*01 positive animals made a robust, early CD8 positive lymphocyte response to an epitope in Tat (STPESANL; FIG. 1A in Example 1). In two of these animals, approximately 10% of their CD8/CD3 positive lymphocytes were directed against this Tat epitope. In vitro culture of PBMC with the STPESANL epitope demonstrated that these CD8 positive lymphocytes killed peptide-pulsed targets, confirming that at least some of these Tat-specific CD8 positive lymphocytes were CTL (FIG. 1B in Example 1).

FIG. 1 describes quantitation of CD8-positive T-lymphocyte responses to various Mamu-A*01-bound peptides. Comparison of CD8-positive T-lymphocyte responses to 6 different epitopes in 10 Mamu-A*01 positive SIV-infected macaques during the first 12 weeks of infection demonstrates a strong CD8-positive T-lymphocyte response to Tat during the acute phase. The relative positions of the CD8 epitopes in the SIV proteins are shown underneath the tetramer histogram. The Mamu-A*01 Tat₂₈₋₃₅ tetramer was initially constructed using an SIVmac251-derived peptide (TTPESANL) (SEQ ID NO: 1). This tetramer detected strong responses during the acute phase of SIV_{mac}239 infected macaques, even though the corresponding SIV_{mac}239 sequence was STPESANL. Subsequent staining with the Tat₂₈₋₃₅ STPESANL (SEQ ID NO: 2) peptide SIV tetramer yielded identical results.

Responses to the well-characterized Gag epitope (CTPYDINQM) (SEQ ID NO: 3) were also high, as previously described. Since 8 of 10 of these animals were previously immunized with DNA and recombinant MVA expressing this Gag epitope, much of these CTPYDINQM responses can probably be attributed to a vaccine effect. However, both Gag- and Tat-specific responses were also detected in two Mamu-A*01 positive macaques that were not vaccinated. In contrast to these two immunodominant responses, the other 4 Mamu-A*01 epitopes did not elicit consistently strong responses during the acute phase.

ii. Bulk Sequencing Detects Variation in the Tat Epitope During the Acute Phase

Of the three strong responses, the frequency of Tat-specific CTL declined more precipitously than did the frequency of CTL recognizing the Gag epitope. We reasoned that this decline could be attributed to viral escape from the Tat-specific CTL. We investigated this possibility by sequencing the 5′ exon of Tat from plasma virus from the ten Mamu-A*01 positive animals. FIG. 2 (Example 1) describes variation present in the SL8 epitope of all 10 Mamu-A*01 positive animals after acute SIV infection using analysis of clones isolated from plasma virus. FIG. 2A (Example 1) describes variation in the SL8 epitope in Mamu-A*01 positive animals infected with molecularly cloned SIV_{mac}239 Nef stop or SIV_{mac}239 Nef open. Limited variation is detectable in the inocula. The predicted amino acid translation of a minimum of 9 clones isolated 6-8 weeks post-infection is shown. The frequency of the epitope variant is shown at the right of the sequence. FIG. 2B (Example 1) depicts little variation outside the 5′ exon of Tat in Mamu-A*01 positive animals. The entire 5′ exon of Tat in two Mamu-A*01 positive animals is shown. Amino acid substitutions accumulate primarily in the STPESANL epitope during the first eight weeks of infection. FIG. 2C (Example 1) examines viral populations within the Mamu-A*01 restricted SL8 epitope evolve rapidly during the acute phase. Predicted amino acid sequences of virus derived 2 weeks, 4 weeks, 6 weeks, and 8 weeks post-infection in animals 96114 and 96118. By 4 weeks post-infection, sequence variation was detectable within the epitope. FIG. 2D (Example 1) describes temporal relationships among viral load, tetramer levels and percent wild-type and escaped virus. Values were averaged for animals 96114 and 96118, illustrating that loss of wild-type virus is coincident with the peak of tetramer levels and with the decline in plasma virus concentrations.

By 8 weeks post-infection, marked variation in the Tat epitope (FIG. 2A in Example 1) was observed in 8 of the 10 animals as detected by bulk sequencing of plasma virus. Surprisingly, this variation was present as early as 4 weeks post-infection in animals challenged with the SIVmac239 nef open molecular clone (FIG. 2A in Example 1).

We then investigated whether these changes in the STPESANL Tat epitope resulted from a mixed population of variants in our inocula. As expected from a molecular clone, there was little variation in this epitope in either of the two inocula (FIG. 2B in Attachment A). Next, we reasoned that these epitope variants might be phenotypic changes that increase virulence. However, only one of eight Mamu-A*O1 negative animals exhibited changes in the Mamu-A*O1-restricted Tat epitope as assessed by direct sequencing (FIG. 2B in Attachment A). Thus, viral escape from the Mamu-A*01-restricted Tat-specific CD8 response appeared to be the most consistent explanation for our findings.

iii. Analysis of Molecular Clones at 8 Weeks Post-Infection Reveals that the Majority of the Replicating Virus in Mamu-A*O1 Positive Animals Encodes Variant CTL Epitopes

Cloning and sequencing of the 5′ exon of Tat from plasma virus at 8 weeks post-infection revealed that RNA extracted from plasma virus from all of the Mamu-A*O1-positive macaques encoded variant Mamu-A*O1-restricted Tat epitopes (FIG. 3A in Example 1). FIG. 3 is a set of six graphs describing CTL analyses of CD8-positive T-lymphocyte cell lines stimulated with SL8 peptide. Cell lines were stimulated with the index peptide (SL8) and autologous B-LCL. After 2 weeks in culture, these T-cell lines were used in CTL analyses at an Effector:Target (E:T) ratio of 25:1 with the index peptide and the variants. Three different dilutions of peptides were tested.

The diversity of the STPESANL CTL epitope in the plasma virus detected by clonal analysis was much greater than that suggested by analysis of the bulk sequencing results. Approximately 75% of clones obtained at 8 weeks post-infection contained variation in the CTL epitope. Indeed in four of the ten Mamu-A*01 positive animals all clones from each animal contained mutations in the Mamu-A*01-restricted STPESANL Tat epitope. Analysis of clones derived from the plasma virus at 8 weeks in the Mamu-A*01-negative animals and the inocula demonstrated limited variation in the STPESANL Tat epitope bound by Mamu-A*01 (FIG. 3A in Example 1). We isolated a single clone from the Nef open inoculum that contained a S→P substitution at P1. This substitution was also found in one of the Mamu-A*01 negative animals. Interestingly, other regions of the 5′ exon of Tat (outside the STPESANL epitope) from four of the Mamu-A*01 negative animals and one of the Mamu-A*01 positive animals (95061) showed variation, consistent with the recognition of these regions by other Tat-specific CTL and their subsequent escape in these animals.

iv. No Evidence for CTL Selection in the Mamu-A*01 Animals in an Overlapping Vpr Open Reading Frame

Serendipitously, the STPESANL epitope in Tat overlaps with a Vpr open reading frame. We were therefore able to determine whether the RNA encoding the Vpr protein had undergone selection. As expected, the Vpr protein encoded by RNA that also coded for the STPESANL epitope showed little or no variation in the Mamu-A*01 positive animals, indicating that selection was specific for the Tat STPESANL epitope. Indeed, there was actually more variation in Vpr in the non-Mamu-A*01 animals than there was in the Mamu-A*01 animals (FIG. 3B in Example 1).

V. Wild-Type Virus Predominates During Peak Viremia at 2 Weeks Post-Infection.

We then performed a time course analysis of viral evolution within the STPESANL Tat epitope in two Mamu-A*01 positive animals. At the peak of viremia, 2 weeks post-infection, Tat-specific CTL responses were barely detectable. FIGS. 4A and B graph the inverse correlation between plasma viral concentrations and peak d_{N} in Tat. Peak d_{N} in Tat was plotted against plasma viral concentrations at peak viremia and at 2 weeks post-peak viremia, revealing a significant inverse correlation between peak d_{N} and viral concentration at 2 weeks post-peak viremia.

No changes in the Mamu-A*01-bound epitope (FIG. 4 in Example 1) were detected by either bulk sequencing or clonal analysis of plasma virus at this time point. At week four, however, variation was apparent in the virus populations of both animals, one week following the highest levels of Tat-specific CTL. The viral sequences within the epitope continued to evolve at six and eight weeks (the latest available time points from these animals). In addition, variants, rather than inoculum sequences, predominated in four animals that have been infected for one year (96031, 95045, 95058, and 95115; data not shown).

vi. No Variation in Other Epitopes in Gag, Env, and Vif Recognized by SIV-Specific CTL.

Finally, we tested whether similar variation was detectable in the other five Mamu-A*01-restricted epitopes recognized by CD8 positive lymphocytes from these animals. In contrast to the variation selected by the Tat-specific CTL, analysis of the epitope in Gag (CTPYDINQM) recognized by the other robust CD8 positive lymphocyte response in these animals showed no variation at 8 weeks post-infection by either bulk sequencing or by clonal analysis. Sequencing of the Gag CTPYDINQM epitope approximately one year post-infection revealed escape in only one of the five Mamu-A*01 positive animals examined (data not shown). Similarly, variation was not detected at eight weeks post-infection within the other four Mamu-A*01 epitopes for which we have tetrameric reagents.

vii. The New Variants in the STPESANL Tat Epitope Diminishes CTL Recognition

To determine whether the observed sequence changes represent viral escape variants, we characterized the functional consequence of the variant epitopes on peptide binding to Mamu-A*O1 and CTL recognition. In vitro peptide binding analyses demonstrated that the new variants of the STPESANL epitope in Tat did not bind to Mamu-A*01 as well as the wild-type peptides did (Table 1 in Example 1). The proline substitution at P1 reduced peptide binding by more than 50%, whereas the leucine substitution at P5 reduced binding by more than 80%. The isoleucine substitution at P2 and the glutamine, arginine and proline substitutions at P8 nearly abrogated binding (>99%). This was not unexpected, since P2 is a secondary anchor and P8 is the carboxy anchor for peptides bound by the Mamu-A*O1 molecule. Similarly, CTL analyses of cell lines generated from the PBMC of several Mamu-A*01 animals stimulated with the STPESANL inoculum peptide recognized all of the new variant epitopes poorly (FIG. 3 in Example 1). Thus, it is likely that the new STPESANL Tat epitope variants diminish the ability of CTL to recognize their targets in vivo.

viii. Hypothesis

Our preliminary data indicates that the traditional course of viral replication in HIV and SIV is actually made up of two different phases. The peak of viremia at 2 weeks after infection is made up of wild-type virus, whereas the virus replicating at the set point is made up largely of virus that has escaped the immune response (FIG. 4 in Example 1). Furthermore, this implies that the cellular immune responses that have selected for the new viral variants have actually destroyed all cells that produce wild-type virus. In this proposal we will explore this radical new hypothesis.

C.2 Avidity

Despite a growing understanding of acute and chronic T-cell responses to HIV and SIV, it remains unknown what constitutes an effective CTL response capable of controlling these infections. We have observed that CTL specific for an epitope in Tat (SL8) select for escape variants during the acute phase of SIV infection. However, acute CTL escape was not induced by a similarly strong CTL response against another well-defined CTL epitope in Gag (CM9). This suggested that CTL specific for this SL8 epitope in Tat were particularly effective at initially controlling the virus. However, the discordance between the ability of the Tat and Gag-specific CTL to exert selective pressure was not understood. We now have data (see Example 2) comparing four SIV-specific CTL responses (in Tat or Nef) capable of inducing acute escape with other CTL responses which do not demonstrate the ability to rapidly escape. CTL lines generated in vitro against each epitope were tested by intracellular cytokine staining (ICS) for their ability to recognize high or low concentrations of each epitope. To compare individual T-cell responses we measured the concentration of peptide required to induce the ½ₘₐₓᵢₘₐₗ percent specific lysis of the ½ₘₐₓᵢₘₐₗ IFN-γ production. The ½ₘₐₓᵢₘₐₗ measurement represents the concentration of peptide in the assay required to induce 50% of the maximal response elicited by any concentration of peptide in the assay. In each case, CD8 cells specific for those epitopes associated with acute escape were capable of recognizing significantly lower concentrations of peptide (½ₘₐₓᵢₘₐₗ concentration of <0.5 nM) compared to those epitopes that escaped late (½ₘₐₓᵢₘₐₗ concentration of >5.0 nM). Similarly, uncultured PBMC specific for the acutely escaped epitopes, which were not altered through in vitro expansion, were superior in their ability to recognize very low concentrations of antigen. These preliminary data indicate that the ability of a CTL response to induce significant selective pressure on the virus during the acute phase of infection is closely linked to the avidity of the CTL response.

i. Strong Acute Phase CTL Responses Induce Mutations in a Tat (SL8) Epitope but not a Gag (CM9) Epitope.

We observed that during the acute phase of SIV infection of 10 Mamu-A*01 rhesus macaques that strong CTL responses were made against two CTL epitopes. One of these epitopes was the well described Gag CM9 response. The other was to an epitope in Tat (SL8). What was striking was that despite similarly strong CTL responses to both of these peptides within the first few weeks after infection, CTL escape was only observed in the Tat SL8 epitope. This suggested to us that there might exist qualitative differences in the CTL responses generated against these two regions of the virus. This could have been a reflection of the fact that the Tat protein is a regulatory protein produced by SIV very early after infection while the structural Gag protein is not produced until later in the viral life cycle. An alternative explanation was that the Tat-specific CTL were of higher avidity than the Gag CTL.

To begin to explore the issue of avidity in vitro CTL lines were generated against both the Gag CM9 and Tat SL8 epitopes and tested in standard 5Cr-release assays. A previous report had illustrated the ability to selectively expand high or low avidity CTL lines depending on the concentration of peptide used to stimulate the cultures. Therefore, cultures were generated using both high (1 μM) and low (1 nM) concentrations of each peptide. 51Cr-release assays revealed that CTL lines specific for the Tat SL8 epitope were capable of recognizing significantly lower concentrations of the appropriate peptide than the Gag CM9 lines (FIGS. 5A and 5B, Example 2). This was true regardless of whether high or low concentrations of peptide were used to generate the cultures. The ½ₘₐₓᵢₘₐₗ specific lysis for the Tat SL8 CTL lines ranged from 0.05-0.10 nM while the Gag CM9 lines ranged from 1-10 nM. Therefore, regardless of the concentration of peptide used to generate the CTL lines the Tat SL8 CTL were capable of recognizing significantly lower concentrations of peptide than the Gag CM9 CTL.

Given that CTL lines require in vitro manipulation we wanted to confirm these differences in peptide recognition using fresh PBMC. Since 51Cr-release assays are not sensitive enough to measure responses when the frequency of CTL are low, intracellular cytokine staining (ICS) for interferon-gamma (IFN-γ) was chosen as a second method to confirm the responses in the CTL lines. Employing flow cytometry, this assay assesses for the production of IFN-γ within a cell in response to an antigen or peptide. Again it was observed that the Tat SL8-specific CD8 T-cells responded to significantly lower levels of peptide than the Gag CM9-specific cells (FIG. 5C, Example 2). The ½ₘₐₓᵢₘₐₗ IFN-γ release for the Tat SL8-specific PBMC was 0.11 nM while the Gag CM9-specific PBMC was 8 nM. The ability of the Tat SL8 CTL to recognize significantly lower concentrations of peptide suggested that these CTL were of higher avidity than the Gag CM9 CTL. Therefore, the ability of a CTL response to induce escape appeared to be associated with the avidity of the CTL response.

ii. Whole Genome Sequencing of SIV in 2 non-A*01 Rhesus Macaques Reveals 2 Additional Acute Escaped CTL Epitopes in Nef.

Since the Tat SL8 epitope was virtually the only epitope to have escaped during the acute phase in Mamu-A*01-positive rhesus macaques we were interested in assessing whether similar acute phase escaped epitopes occurred in non-A*01 macaques. Bulk whole genome sequencing of plasma taken at 4 weeks post-infection revealed that a number of different epitopes escaped during the acute phase of infection in non-A*01 macaques. Two of these epitopes which exhibited particularly dramatic mutations were selected to be sequenced in more detail using molecular clones which revealed a significant accumulation in mutations in each epitope (Table 3, Example 2). These epitopes are listed in Table 2 as Nef_YY9 and Nef_GL9 (Table 2 in Example 2). This confirmed that the Tat SL8 mutation observed during the acute phase of SIV infection in Mamu-A*01 rhesus macaques was not limited to Tat or to Mamu-A*01 macaques.

iii. Optimal Identification of the 2 Acute Escaped CTL Epitopes.

In order to determine whether cellular immune responses were associated with the 2 new acute mutations observed in Nef, an ELISPOT assay was employed. This assay measures for the production of IFN-γ by PBMC stimulated with various peptides as a measure of an immune response recognizing an foreign antigen. 15-amino acid peptides spanning each of the escaped epitopes were tested in animals from which the escaped epitopes were originally identified. These assays confirmed that cellular responses were associated with the regions of the virus in which these escaped epitopes were identified. In order to determine the avidity of a T-cell response to a given epitope it is necessary to definitively know the optimal minimal length of the CTL epitope in question. To accomplish this in vitro CTL lines were generated against each escaped epitope from PBMC of the animal from which the escaped epitopes were originally identified. These CTL lines were then tested in intracellular cytokine staining assays with peptides of 8-, 9-, or 10-amino acids in length overlapping the region of SIV in question. This assay determines which of the overlapping peptides is best able to induce an immune response, in this case the production of an antiviral protein interferon-gamma (IFN-γ). Since non-optimal peptides can induce significant responses when tested at very high concentrations it is necessary to test each of the potential peptides in this assay at several peptide dilutions. The optimal peptide is then identified as the peptide which stimulates significant IFN-γ production even at relatively low peptide concentrations. This assay defined the minimal optimal length of each of these epitopes (Table 3, FIG. 6 in Example 2).

iv. The Two New Acute Escaped Epitopes Represent High Avidity CTL Epitopes.

Once the optimal length of the 2 nef epitopes was identified we were able to assess whether the T-cell responses for inducing these mutations also represented high avidity CTL epitopes. Using the data from FIG. 6 which defined the optimal CTL epitope for each response, the concentration curve which corresponds to the optimal epitope also represents the concentration curve used to determine the avidity of the CTL response to that epitope. Replotting of these concentration curves to these optimal epitopes along with Tat SL8 reveals that the 2 Nef epitopes along with the Tat SL8 epitope represent high avidity CTL responses with ½ₘₐₓᵢₘₐₗ IFN-γ production occurring at <0.5 nM (FIG. 7 in Example 2). Now that these responses have been defined as minimal epitopes and their avidity has been confirmed in CTL lines it will be necessary to confirm their avidity in PBMC which have not undergone in vitro manipulations. Frozen PBMC taken early after infection at a time point when the frequency of these CTL would have still been high enough to detect these responses will be used to confirm this.

v. Defining the Avidity of CTL Responses Which do not Induce Acute Escape.

While we have defined that the avidity of the Gag CM9 CTL response which does not induce acute escape is low, it will be necessary to define the avidity of a number of epitopes that do not escape during the acute phase. To this end we have preliminary data testing the avidity of 3 additional CTL epitopes, Nef_AL11, Env_KL9, and Env_GI8 (Table 2, in Example 2). As determined by intracellular cytokine staining each of the epitopes represented low avidity CTL responses with ½ₘₐₓᵢₘₐₗ IFN-γ production at >5.0 nM (FIG. 8 in Example 2). This preliminary data confirms that other CTL epitopes that do not induce acute escape are also of low avidity.

vi. High Avidity CTL Epitopes can also be Identified in Uninfected Individuals

High avidity CTL responses can also be identified in uninfected individuals who have been vaccinated against HIV or SIV. The ICS profiles for interferon-gamma (IFN-γ) of low and high affinity CTL are different. High avidity CTL stain much brighter with anti-cytokine antibodies such as IFN-g and tumor necrosis factor-alpha (TNF-α) after stimulation with the same peptide concentration (FIG. 9, Example 2) compared with low avidity CTL. High avidity CTL also possess a greater degree of CD8 down-regulation in comparison to low avidity CTL after stimulation with their cognate ligand (peptide) at the same concentration (FIG. 9, Example 2). These staining profiles are seen both in infected animals and in SIV-vaccinated animals. Therefore, in the setting of HIV we could potentially use this additional approach to define high avidity CTL responses in individuals currently enrolled in various HIV vaccine clinical trials.

vii. Hypothesis

Our preliminary data suggests that the potential of a CTL response to induce a selective pressure capable of inducing rapid escape in SIV is directly linked to the avidity of the CTL response. Previous publications studying viral infections other than HIV or SIV indicate that high avidity CTL responses are significantly more capable of controlling a viral infection. Unfortunately, due to the capability of HIV and SIV to mutate in order to evade host immune responses identifying these high avidity CTL responses in HIV infected patients will be difficult since the immune response to these epitopes will rapidly decline following mutation of the virus and PBMC from the acute phase are rarely obtained. Viral sequencing from samples taken during the peri-acute phase, however, will retain these mutations making identification of these acute escaped responses possible. Therefore, the identification of these acute phase escaped CTL epitopes as high avidity CTL responses validates the importance of these unique CTL responses in controlling a viral infection such as HIV.

### EXAMPLES

### Example 1

Materials and Methods

Tetramer Analysis

Soluble tetrameric Mamu-A*01 MHC class I/SIV peptide complexes were constructed as previously described (Allen, T. M., et al., J. Virol. Submitted, 2000; Altman, J. D., et al., Science 274:94-96, 1996). Background tetramer staining of fresh, unstimulated PBMC from naive Mamu-A*01^{+ animals was routinely less than} 0.08%.

Amplification of Viral RNA from Plasma and Sequence Detection

SIV plasma virus sequence was obtained as previously described (Evans, D. T., et al., Nat. Med. 5:1270-1276,1999). The primers used to amplify cDNA encoding the Mamu-A*01 Tat epitope included SIV 6511-F (5′TGATCCTCGCTTGCTAACTG3′ (SEQ ID NO: 5)) and 6900-R (5′AGCAAGATGGCGATAAGCAG3′ (SEQ ID NO: 4)). The cloned inserts were isolated and sequenced as described above using the SIV 6511-F and SIV 6900-R primers. Seven overlapping PCR primer pairs were used to amplify cDNA spanning the entire SIV genome. Primer sequences are shown in supplementary data, FIG. 2B. The PCR products were directly sequenced off both cDNA strands. Overlapping sequence between the primers linked together sequences from the individual RT-PCR reactions. Sequence editing and finishing was performed using Auto Assembler v2.1 on the Macintosh. Nucleotide and predicted amino acid sequences were aligned using MacVector 4.1 (Oxford Molecular).

Mamu-A*01 Binding Assay

Quantitative assays for the binding of peptides to soluble Mamu-A*01 molecules on the basis of the inhibition of binding of a radiolabeled standard probe peptide to detergent-solubilized MHC molecules were performed as previously described (Allen, T. M., et al., J. Immunol. 160:6062-6071, 1998). A position 1 C→A analog of the SIV gag 181-190 peptide (ATPYDINQML) was utilized as the radiolabeled probe. In the case of competitive assays, the concentration of peptide yielding 50% inhibition of the binding of the radiolabeled probe peptide was calculated. Peptides were initially tested at one or two high doses. The IC₅₀ of peptides yielding positive inhibition were then determined in subsequent experiments, in which two to six further dilutions were tested, as necessary. Since under the conditions to be utilized, where [label]<[MHC] and IC₅₀ ≧[MHC], the measured IC₅₀ values are reasonable approximations of the true Kd values. Each competitor peptide was tested in two to four completely independent experiments. As a positive control, in each experiment the unlabeled version of the radiolabeled probe was tested and its IC₅₀ measured.

Generation of in Vitro Cultured CTL Effector Cells

CTL cultures were established from peripheral blood samples of SIV-infected rhesus macaques drawn in EDTA tubes and CTL were cultured and assayed as previously described (Evans, D. T., et al., supra, 1999).

Animals, Viruses, and Infections

Rhesus macaques used in this study were identified as Mamu-A*01+ by PCR-SSP and direct sequencing as previously described (Knapp, L. A., et al., Tissue Antigens 50:657-661,1997). All rhesus macaques used in this study were Mamu-A*01+, with the exception of animals 95003, 95112, 96020, 96081, 96093, 96072, 96104 and 96113. Rhesus macaques, 95045, 96031, 95058, 96118, 96123, 95061, 96114 and 94004 were vaccinated with a DNA/MVA regimen expressing the Gag181-189 peptide (CTPYDINQM) (Allen, T. M., et al., J. Immunol. 164:4968-4978, 2000). The Mamu-A*01 positive macaques 95114 and 95115 were not vaccinated prior to challenge. All rhesus macaques were infected intrarectally with a molecularly cloned virus; SIVmac239 (Regier, D. A. and Desrosiers, R. C., AIDS Res. Hum. Retroviruses 6:1221-1231, 1990) (either Nef stop [95045, 96031, 95058, 95114, 95115, 95003 and 95112] or Nef open [99118, 96123, 95061, 96114, 94004, 96020, 96081, 96093, 96072, 96104, and 96113]). Plasma viral concentrations were measured by bDNA analysis (Chiron). This stock was amplified on rhesus PBMC only. SIV-infected animals were cared for according to an experimental protocol approved by the University of Wisconsin Research Animal Resource Committee.

Statistical Analysis of Sequence and Plasma Virus Concentration Data

Numbers of synonymous nucleotide substitutions per synonymous site (d_{S}) and of nonsynonymous nucleotide substitutions per nonsynonymous site (d_{N}) were estimated following the Nei and Gojobori method (Nei, M. and Gojobori, T., Molec. Biol. Evol. 3:418-426, 1986). For the sample of viral sequences taken from a given animal, the means of d_{S} and d_{N} were computed for (a) all pairwise comparisons between each viral sequence and viral sequences sampled from the inoculum; and (b) all pairwise comparisons among viral sequences within the sample. These quantities were computed for the Tat₂₈₋₃₅ epitope and the remainder of the 98-codon portion of Tat that was sequenced. To evaluate the statistical significance of the difference in peak and post-peak viremia between macaques with high and low d_{N} in Tat, we compared the natural log (i.e., log base e) plasma virus concentrations among animals with high and low d_{N}. Taking the logs greatly improved the fit of the data to the assumptions of the statistical models employed (i.e., normality, homoscedasticity in a multivariate test). We also used MANOVA which is dependent on fewer assumptions than the repeated measures ANOVA.

TABLE 1

Peptide Binding of Mutant Tat Epitopes to the Rhesus MHC Class I
Molecule Mamu-A*01

| | | | |
|---|---|---|---|
| | Relative Binding | | % Binding |
| ^{a}IC₅₀ | Average ^{b} | ± s.d. | reduction^{c} |

| | | | | | |
|---|---|---|---|---|---|
| Tat₂₈₋₃₅ Epitope (index) | STPESANL | 43 | 1 | 0 | 0 |
| Variant | P------ | 100 | 0.43 | 0.025 | 67 |
| Variant | -I---- | 4611 | 0.009 | 0.001 | >99 |
| Variant | ----L-- | 281 | 0.155 | 0.03 | 85 |
| Variant | ------Q | 13175 | 0.003 | 0.0004 | >99 |
| Variant | ------P | 4573 | 0.009 | 0.004 | >99 |
| Variant | ------R | 15091 | 0.003 | 0.0012 | >99 |

^{a 125}labeled peptide: ATPYDINQML
^{b}Relative peptide binding is expressed relative to the binding affinity of the Tat₂₈₋₃₅ STPESANL to the rhesus macaque MHC class I molecule Mamu-A*01.
^{c}Percent binding reduction is calculated as (1.0-relative binding average)×100.

Results

The strongest CD8-positive T lymphocyte responses to HIV and SIV are observed in the first few weeks of infection, coincident with the initial decline in plasma viremia. We hypothesized that viral escape might occur from immune responses that exert selective pressure during this acute phase of infection. To test this hypothesis, we examined viral evolution in the face of CD8-positive T-lymphocyte responses during infection of 18 rhesus macaques with molecularly cloned SIV.

Every animal (10/10) expressing the rhesus MHC class I molecule Mamu-A*01 made CD8-positive T lymphocyte responses to a newly defined epitope in Tat₂₈₋₃₅ STPESANL (Allen, T. M., et a., J. Virol., supra, 2000)(SL8, FIG. 1) which peaked between three and 4 weeks post-infection. In two of these animals, as many as 10% of their CD8/CD3-positive lymphocytes recognized this Tat epitope. However, the frequency of Tat-specific lymphocytes declined precipitously after the acute phase (FIG. 1). We reasoned that this decline might be the result of viral escape from these Tat-specific responses. We investigated this possibility by sequencing the 5′ exon of Tat using virus derived from the ten Mamu-A*01-positive animals. By eight weeks post-infection, a high frequency of amino acid substitution was observed in the SL8 epitope (FIG. 2A). Eighty-six percent of clones contained variation in the CD8-positive T lymphocyte epitope at this time point. In five of the ten Mamu-A*01-positive animals, all sequenced clones contained mutations in the Mamu-A*01-restricted SL8 epitope. By contrast, little amino acid variation was observed outside this SL8 epitope in Mamu-A*01-positive animals (FIG. 2B).

We then investigated whether these changes in the SL8 epitope resulted from a mixed population of variants in our inocula or whether they were selected for increased replicative fitness in Mamu-A*01-negative animals. As expected from a molecular clone, there was little variation in this epitope in either of the two inocula (FIG. 2A). Additionally, only one of eight Mamu-A*01 negative animals exhibited changes in the SL8 epitope. Thus, viral escape from the Mamu-A*01-restricted Tat-specific CD8-positive T lymphocyte responses appeared to be the most consistent explanation for our findings.

We then performed a time course analysis of viral evolution within the SL8 epitope and sequenced the entire virus after the acute phase in two of the Mamu-A*01-positive animals. At peak viremia, at 2 weeks post-infection, Tat-specific CTL responses were barely detectable and no changes in the Mamu-A*01-bound Tat epitope were present (FIGS. 2C and D). After resolution of peak viremia, at three weeks post-infection, Tat-specific CD8-positive T lymphocytes were at their highest level. One week later, extensive variation was apparent in the virus populations of both animals (FIGS. 2C and D). Furthermore, direct sequencing of the open reading frames of the entire virus at 4 weeks post-infection revealed only a single site of viral nucleotide diversity in the SL8 epitope in animal 96118. In animal 96114 there were three sites of viral nucleotide diversity, one of which was in the SL8 epitope, and the other two in Rev and Env. In animal 96118 the nucleotide substitution in RNA encoding the SL8 epitope caused a change in the overlapping reading frame of Vpr. In 96114 the change in Rev also caused a substitution in the overlapping open reading frame of Env. This Rev replacement is seen in most animals infected with this viral clone and appears to be selected for increased viral fitness. Analysis of the additional replacement in 96114 in Env by IFN-γ ELISPOT assays of CD8 and CD4 lymphocytes, however, failed to conclusively show that this region contained any T-cell epitopes.

To determine whether the observed sequence changes in the SL8 epitope indeed represent viral escape variants, we characterized the functional consequences of the predominant variant epitopes on peptide binding to Mamu-A*01 and on CTL recognition. In vitro peptide binding analyses demonstrated that the new variants of the SL8 epitope did not bind to Mamu-A*01 as well as the wild-type peptide (Table 1). The substitutions of proline at P1 and leucine at P5 reduced peptide binding by more than 50% and 80%, respectively. The isoleucine substitution at P2 and the glutamine, arginine and proline substitutions at P8 abrogated binding (>99% reduction). Since P2 is a secondary anchor and P8 is the carboxy anchor (Allen, T. M., et al., supra, 1998) for peptides bound by the Mamu-A*01 molecule, substitutions at anchor residues are expected to have the most profound effect on peptide binding. Similarly, analyses of CTL lines generated from the PBMC of several Mamu-A*01-positive animals stimulated with the SL8 index peptide poorly recognized the new variant epitopes (FIG. 3). Interestingly, variant peptides with the P5 leucine substitution were the least efficient at sensitizing targets for CTL lysis, suggesting that this P5 mutation was likely interfering with TCR recognition. Therefore, it seems likely that the new variants either reduced the amount of Tat-derived peptide MHC class I complexes on the cell surface or reduced the ability of these complexes to be recognized by the T-cell receptor (Price, G. E., et al., J Exp. Med. 191:1853-1867, 2000).

To test the hypothesis that viruses with amino acid replacements within the SL8 epitope are favored by natural selection, we compared the number of synonymous nucleotide substitutions per synonymous site (d_{S}) and the number of nonsynonymous nucleotide substitutions per nonsynonymous site (d_{N}) in the epitope and the remainder of the sequence. In the SL8 epitope region of the virus from Mamu-A*01-positive animals, mean d_{N} was significantly higher than mean d_{S} both for comparisons between samples and the inoculum (d_{N}=5.7±0.4; d_{S}=0.4±0.4, P<0.001) and in comparisons within samples (d_{N}=7.3±1.0; d_{S}=0.7±0.7, P<0.001; supplementary data; Table 1). Mean d_{N} values in the SL8 epitope from Mamu-A*01-positive animals were almost sixty times the corresponding values for Mamu-A*01-negative animals (d_{N}=5.7 in Mamu-A*01 positive animals; d_{N}=0.1 in Mamu-A*01 negative animals). Since a pattern of d_{N}>d_{S} is not expected under neutral evolution (Wolinsky, S. M., et al., Science 272:537-542,1996; Kimura, M., Nature 267:275-276,1977; Hughes, A. L. and Nei, M., Nature 335:167-170,1988), this result strongly implies that amino acid replacements in the SL8 epitope are favored by positive Darwinian selection.

Interestingly, the 5′ exon of four of the eight Mamu-A*01-negative animals showed patterns of variation suggestive of escape from other Tat-specific cellular immune responses (Supplemental Data FIGS. 1 and 3). We, therefore, explored the possibility that animals with little evidence for selection in Tat should have higher plasma virus concentrations than animals with evidence for increased d_{N} in Tat. Since the 18 animals in our cohort were originally part of a vaccine study (Allen, T. M,. et al., supra, 2000), we excluded the 8 vaccinated Mamu-A*01-positive animals from this analysis. The two naïve Mamu-A*01 positive and four of the naïve Mamu-A*01 negative animals exhibited evidence of increased d_{N} peaks within the 5′ exon of Tat, whereas four naïve animals revealed little evidence of increased d_{N} in Tat. Averaging the plasma virus concentrations of these two groups of animals showed a significant difference of at least one log (p=0.008) between the plasma virus concentrations at weeks 2, and 4 post peak viremia of animals with high and low d_{N} in Tat. Similarly, a significant inverse correlation was observed between peak d_{N} and viral load 2 weeks (p=0.007), 4 weeks (p=0.008) and 8 weeks (p=0.048) post-peak viremia (FIG. 4). Of the four animals with no evidence for high d_{N} in Tat, two rapidly progressed to sAIDS and had SIV plasma virus concentrations in excess of 100×10⁶ copies/ml within six months of infection. Therefore, animals with evidence of increased d_{N} in Tat may have controlled wild-type virus better than those with less selective pressure on Tat.

Vaccine-induced cellular immune responses against proteins expressed early in the viral life cycle may be better able to control HIV and SIV replication than responses directed against proteins that are expressed later in the viral life cycle. Viral escape from Tat-specific CD8-positive T lymphocytes occurred with kinetics similar to those seen during the emergence of drug resistant mutants (Coffin, J. M., Science 267:483-489, 1995). In five of ten Mamu-A*01-positive animals, all clones isolated from plasma at six to eight weeks post-infection contained mutations in the Mamu-A*01-restricted SL8 epitope. This implies that Tat-specific CD8-positive T lymphocytes efficiently controlled replication of the original wild-type inoculum virus in these five animals. Responses directed against early proteins such as Tat may be particularly effective at controlling initial virus replication, since Tat and Rev are the only two viral proteins produced before Nef down-regulates MHC class I molecules (Collins, K. L., et al., Nature 391:397-401, 1998). Tat-specific CTL may, therefore, be potent inhibitors of early viral replication, whereas CTL directed against peptides derived from other viral proteins may find few MHC class I/peptide complexes on the cell surface later in the course of the viral life cycle. The differences between the Gag and Tat-specific CTL in their ability to exert selective pressure favoring viral escape are intriguing. Understanding the qualitative differences between these CTL which account for these characteristics will be an important issue in the design of an effective HIV vaccine. Interestingly, vaccination of non-human primates with either Tat protein (Cafaro, A., et al., Nat. Med. 5:643-650, 1999; Pauza, C. D., et al., Proc. Natl. Acad. Sci. USA 97:3515-3519, 2000) or recombinant viruses expressing Tat and Rev (Osterhaus, A. D. M. E., et al., Vaccine 17:2713-2714,1999) have reduced initial virus replication. In these studies it is possible that Tat-specific CD8-positive T lymphocyte responses played a role in reducing the initial peak of viral replication characteristic of the acute phase.

The information presented above in Example 1 was also disclosed in Allen, et al., Nature 407:386-390 (2000), incorporated by reference as if fully set forth herein.

### Example 2

Materials and Methods

Animals and Infections

Rhesus macaques used in this study were identified as Mamu-A*01, -A*02, -A*11, -B*03, or -B*17+by PCR-SSP and direct sequencing as previously described (Knapp, L. A., et al., Tissue Antigens 50:657,1997). Animal 96118 was vaccinated with a DNA/MVA regimen expressing the Gag_{—CM}9 peptide (Allen, T. M., et al., J. Immun. 164:4968, 2000). Animals 96118, 1975, 96072, 95084, and 96081 were infected intrarectally with a molecularly cloned virus; SIVmac239. This stock was amplified on rhesus PBMC only. Animal 95027 was infected intravenously with 40 tissue culture infectious doses 50% (TCID₅₀) of a heterogeneous SIV stock (originally provided by R. C. Desrosiers, Harvard University and New England Regional Primate Research Center). The stock was amplified by growth on rhesus PBMC with a final passage on CEM×174 cells to increase titers (Trivedi, P., et al., J. Virol. 68:7649,1994; Pauza, C. D., et al., J. Med. Primatol. 22:154, 1993). SIV-infected animals were cared for according to an experimental protocol approved by the University of Wisconsin Research Animal Resource Committee. Animals were maintained in accordance with the NIH Guide to the Care and Use of Laboratory Animals, and under the approval of the University of Wisconsin Research Animal Resource Center (RARC) review committee.

Isolation of PBMC

PBMC were isolated from EDTA or heparin-treated whole blood using Ficoll/diatrioate gradient centrifugation. Cells were then washed twice in R10 media (RPMI 1640 supplemented with penicillin (50 U/ml), streptomycin (50 μg/ml), L-glutamine (2 mM), and 10% FBS (Biocell, Carson, Calif.)).

Generation of in Vitro Cultured CTL Effector Cells

CTL cultures were established from EDTA or heparinized-treated peripheral blood samples as previously described (Allen, T. M., et al., J. Immunol. 160:6062, 1998). Briefly, Ficoll-Hypaque separated PBMC were stimulated 1:1 with 5×10⁶ γ-irradiated (3000 rad) autologous B-LCLs pulsed with the appropriate peptide (1 μM unless otherwise noted) in R10 medium. Cultures were supplemented with R10 containing 20 U/ml rIL-2, a gift from Hoffman-LaRoche (Nutley, N.J.). On day 7, viable cells were restimulated and again expanded in the presence of rIL-2. CTL activity was assessed after 14 days of culture in a standard ⁵¹Cr-release assay. Peptides were obtained from the University of Wisconsin Biotechnology Center (Madison, Wis.) as desalted products. Lyophilized aliquots were resuspended in HBSS with 10% DMSO (Sigma) to a final concentration of 1 mg/ml.

Cytotoxicity Assays

SIV-specific CTL activity was assessed using a standard ⁵¹Cr-release assay (Allen, T. M., et al., supra, 1998). Briefly, autologous B-LCL targets were pulsed with SIV peptides (varying concentrations) or an irrelevant influenza NP peptide (SNEGSYFF) and 80 μCi Na₂⁵¹CrO₄ (New England Nuclear Life Sciences Products) for 1.5 hours. Target cells (5×10³) were incubated for 5 hours with CTL effectors at E:T ratios ranging from 10:1 to 20:1. CTL activity was calculated from the CPM present in harvested supernatants using the formula: Percent specific release=(Experimental release−Spontaneous release)/(Maximal release−Spontaneous release)×100. The reported % specific lysis represents the ⁵Cr-release from the Mamu-A*01 peptide pulsed targets minus the ⁵¹Cr-release from target cells pulsed with the irrelevant influenza NP peptide (SNEGSYFF). Spontaneous release was always less than 20% of maximal release.

Intracellular IFN-γ Staining

For in vitro stimulated CTL cultures, 2×10⁵ cells were incubated at 37° C. for 1.5 hours with PMA/lonomycin (50 ng/ml and 1 μg/ml, respectively), varying concentrations of peptide, or a control influenza peptide (SNEGSYFF) in the presence of autologous B-LCL (1×10⁵) as antigen presenting cells. For whole PBMC 0.5×10⁶−1.0×10⁶ thawed PBMC were treated with 1 μl of anti CD28 (BectonDickenson; cat#348040) and 1 μl of anti CD49d (Pharmingen; cat #31471A) in place of the BLCL. Cells were then treated with 10 μg/ml of Brefeldin A (BFA) to inhibit protein trafficking and incubated a further 4 hours (CTL lines) to 5 hours (PBMC) at 37° C. Cells were then washed twice with FACS buffer (PBS+2% FCS) and stained with 6 μl of CD8α-PerCP (BectonDickenson, clone SK1, cat#347314). After fixation with PFA (overnight), cells were washed twice with FACS buffer and treated with 150 μl, of permeablization buffer (0.1% saponin in FACS buffer) for 5 minutes at room temperature. Cells were washed once more with 0.1% saponin and then incubated in the dark for 50 minutes with 1 μl of anti-human IFN-γ-FITC mAb (Pharmingen; clone 4S.B3; cat#18904A) and 1 μl, anti-human TNFα-PE (Pharmingen MAb 11, cat#18645A). Finally, cells were washed twice with 0.1% saponin-buffer and a 100 μl cell suspension was fixed with 200 μl of 2% paraformaldehyde (PFA).

Results

Strong Acute Phase CTL Responses to SIV Induce Mutations in an Epitope in Tat but not Gag.

We previously observed that CTL specific for an epitope in Tat (SL8) select for escape variants during the acute phase of SIV infection while acute escape was not induced by a similarly strong acute phase CTL response against an epitope in Gag (CM9). This suggested that CTL specific for the SL8 epitope in Tat were particularly effective at initially controlling the virus.

To begin to explore the issue of avidity in vitro CTL lines were generated against both the Gag CM9 and Tat SL8 epitopes and tested in standard ⁵¹Cr-release assays. A previous report had illustrated the ability to selectively expand high or low avidity CTL lines depending on the concentration of peptide used to stimulate the cultures (Alexander-Miller, M. A., et al., Proc. Natl. Acad. Sci. USA 93:4102-4107, 1996). Therefore, cultures were generated using both high (1 μM) and low (1 nM) concentrations of each peptide. ⁵¹Cr-release assays revealed that CTL lines specific for the Tat SL8 epitope were capable of recognizing significantly lower levels of cognate peptide than the Gag CM9 lines (FIGS. 5A and 5B). This was true regardless of whether high or low concentrations of peptide were used to generate the cultures. The ½ₘₐₓᵢₘₐₗ specific lysis for the Tat SL8 CTL lines ranged from 0.05-0.10 nM while the Gag CM9 lines ranged from 1-40 nM. Therefore, regardless of the concentration of peptide used to generate the CTL lines the Tat SL8 CTL were capable of recognizing significantly lower concentrations of peptide than the Gag CM9 CTL.

Given that CTL lines require in vitro manipulation we wanted to confirm these differences in peptide recognition using fresh PBMC. Since ⁵Cr-release assays are not sensitive enough to measure responses when the frequency of CTL are low, intracellular cytokine staining (ICS) for interferon-gamma (IFN-γ) was chosen as a second method to confirm the responses in the CTL lines. This assay assesses for the production of IFN-γ within a cell in response to an antigen or peptide. Again it was observed that the Tat SL8-specific CD8 T-cells responded to significantly lower levels of peptide than the Gag CM9-specific cells (FIG. 5C). The ½ₘₐₓᵢₘₐₗ IFN-γ release for the Tat SL8-specific PBMC was 0.11 nM while the Gag CM9-specific PBMC was 8 nM. The ability of the Tat SL8 CTL to recognize significantly lower concentrations of peptide suggested that these CTL were of higher avidity than the Gag CM9 CTL. Therefore, the ability of a CTL response to induce escape appeared to be associated with the avidity of the CTL response.

Whole Genome Sequencing of SIV in non-A*01 Rhesus Macagues Reveals 2 Additional Acute Escaped CTL Epitopes in Nef and Vpr.

In order to determine whether this association between high avidity CTL and acute escape represented a repeatable phenomenon it was necessary to extend these findings to other acute escaped epitopes. Two additional acute CTL epitopes that accumulate amino acid replacements with similar kinetics to the Tat SL8 epitope were recently identified (Table 3). These additional acute phase escaped epitopes were defined in Mamu-A*01-negative macaques in the Nef protein (Table 3). For comparison, we selected 4 SIV-specific CTL epitopes, in addition to the Gag CM9 epitope, to which CTL escape had not been observed to occur sooner than 6 months post-infection (Table 2). While some of the chronically escaping CTL epitopes have not been extensively studied due to their low frequency MHC class I molecules, we have not observed any of the Mamu-A*01-restricted chronic CTL epitopes to have escaped in Mamu-A*01 animals prior to 1 year post-infection.

Fine Mapping of Two New Acute Phase Escaped SIV CTL Epitopes.

Since the peptide dilutions assays used to determine the avidity of a CTL response require knowledge of the minimal optimal peptide each of these epitopes was fine mapped. To accomplish this in vitro CTL lines were generated against each escaped epitope from PBMC of the animal from which the escaped epitopes were originally identified. These CTL lines were then testing in intracellular cytokine staining (ICS) assays with peptides of 8-, 9-, or 1 0-amino acids in length overlapping the region of SIV which accumulated amino acid replacements. Since non-optimal peptides can induce significant responses when tested at very high concentrations it is necessary to test each of the potential peptides in this assay at several peptide dilutions. As expected, many of the peptides demonstrated good responses at high peptide concentrations. However, when these peptides were diluted out to concentrations as low as 0.05 nM only a single peptide for each CTL line demonstrated significant reactivity, defining the optimal peptides associated with the CTL response (FIG. 6). These epitopes are referred to as Nef YY9 and Nef GL9.

The Two New Acute Escaped Epitopes Represent High Avidity CTL Responses.

Once the optimal lengths of the two acute escaped CTL epitopes was identified it was possible to assess whether the T-cell responses for inducing these mutations also represented high avidity CTL epitopes. The concentration curves that corresponded to the optimal epitopes in FIG. 6 were replotted along with the Tat SL8 epitope. FIG. 7 illustrates that the CTL lines generated against each acute escaped CTL epitope were capable of recognizing extremely low concentrations of peptide with ½ₘₐₓᵢₘₐₗ IFN-γ production levels of <0.10 nM. Therefore, the two Nef epitopes YY9 and GL9 represent high avidity CTL responses similar to those of Tat SL8. It will also be necessary to assess the avidity of these CTL responses using whole PBMC.

In comparison, it was necessary to determine the avidity of PBMC specific for 3 other late escaping epitopes. These epitopes had previously been defined as having escaped in rhesus macaques infected with SIVmac239, although these escapes did not occur before 1 year post infection (Table 2). Using ICS it was observed that each of the Nef_AL11, Env_KL9, and Env GI8 epitopes demonstrated low avidity, in fact even lower than for that of Gag CM9 (FIG. 9). This will be repeated for CTL lines specific for each of these epitopes.

High Avidity CTL Epitopes Can Also be Identified in Uninfected Individuals

High avidity CTL responses can also be identified in uninfected individuals who have been vaccinated against HIV or SIV. The ICS profiles for interferon-gamma (IFN-γ) of low and high affinity CTL are different. High avidity CTL stain much brighter with anti-cytokine antibodies such as IFN-g and tumor necrosis factor-alpha (TNF-α) after stimulation with the same peptide concentration (FIG. 9, Example 2) compared with low avidity CTL. High avidity CTL also possess a greater degree of CD8 down-regulation in comparison to low avidity CTL after stimulation with their cognate ligand (peptide) at the same concentration (FIG. 9, Example 2). These staining profiles are seen both in infected animals and in SIV-vaccinated animals. Therefore, in the setting of HIV we could potentially use this additional approach to define high avidity CTL responses in individuals currently enrolled in various HIV vaccine clinical trials.

Discussion

Despite a growing understanding of acute and chronic T-cell responses to HIV and SIV, it remains unknown what constitutes an effective CTL response capable of controlling these infections. We have now observed that CTL specific for epitopes which select for escape variants during the acute phase of SIV infection demonstrate significantly higher avidity than CTL specific for epitopes that do not induce escape. These data indicate that the ability of a CTL response to induce significant selective pressure on the virus during the acute phase of infection is closely linked to the avidity of the CTL response. Together these findings support the concept that CTL specific for epitopes such as Tat SL8 should be particularly effective at controlling the virus. We now have data comparing three SIV-specific CTL responses in Tat and Nef capable of inducing acute escape. Further sequencing of virus from the acute phase of SIV infection should enable the definition of additional high avidity CTL epitopes.

Previous publications studying viral infections other than HIV or SIV indicate that high avidity CTL responses are significantly more capable of controlling a viral infection. Our ability to define two independent yet supporting approaches to identify these unique CTL responses capable of exerting pressure upon SIV may be particularly important for investigating these unique CTL responses in HIV. These experiments were feasible in the SIV-infected rhesus macaque model as there is a comprehensive understanding of the route, dose, day of infection and sequence of the virus infecting these animals. Furthermore, once the epitopes that escaped rapidly were identified, obtaining acute PBMC samples in which these CTL responses were high enough in frequency to examine was possible. In the setting of HIV infected patients, the dose, day of infection and sequence of the virus is often completely unknown and obtaining PBMC or viral samples from early after infection is difficult. However, having two independent methods available to identify and validate such unique acute CTL responses in HIV should facilitate their identification in HIV infected patients.

Our findings indicate that a particular qualitative parameter of CTLs (avidity) is associated with the ability of a CTL response to induce substantial selective pressure upon SIV during the acute phase. This suggests that particular mutations do not occur more easily than others simply due to the ability of certain regions of the virus to more easily tolerate change. It may be difficult to identify rapidly escaping CTL epitopes in HIV-infected individuals due to a lack of acute phase samples and the uncertainty of the infecting strain. However, identification of an easily quantifiable immune parameter (avidity), which is associated with acute escape, may provide an additional approach to identify those HIV-specific CTL epitopes capable of inducing significant selective pressure. Such epitopes may represent a critical component of an effective HIV vaccine.

TABLE 2
Acute Escaping and Non-Escaping SIV-specific CTL epitopes.

| SEQ ID | | MHC | Acute | Earliest | |
|---|---|---|---|---|---|
| NO: | CTL Epitope | Restriction | Escape | Escape | Animal |
| 6 | Tat_SL8 (STPESANL) | A*01 | Yes | 4 wks p.i. | 1975+ |
| 7 | Nef_YY9 (YTSGPGIRY) | A*02 | Yes | 4 wks p.i. | 95084 |
| 8 | Nef_GL9 (GLDKGLSSL) | ? | Yes | 4 wks p.i. | 96081 |
| 9 | Gag_CM9 (CTYPDINQM) | A*01 | No | >12 mo. p.i. | 1975+ |
| 10 | Nef_Al11 (ARRHRILDIYL) | B*03 | No | 25-41 wks p.i. | 95027/88090 |
| 11 | Env_KL9 (KRQQELLRL) | B*03 | No | >25 mo. p.i. | 95027/88090 |
| 12 | Env_GI8 (GDYKLVEI) | A*11 | No | >12 mo. p.i. | 95027/89104 |

TABLE 3

Rapid Viral Escape Detected by 4 Weeks Post-Infec-
tion in SIV-Infected Rhesus Macaques

| | | | |
|---|---|---|---|
| SEQ ID NO:13 | 94004 | STPESANL | (A*01) |
| | Tat | | |
| | | ........Q | (2/10) |
| | | ...G.... | (2/10) |
| | | ........R | (2/10) |
| | | ........P | (2/10) |
| | | .I....... | (1/10) |
| | | ..L...... | (1/10) |
| SEQ ID NO:14 | 95084 | PDWQDYTSGPGIRYP | (A*02) |
| | Nef 63 | | |
| | | .........L........ | (5/10) |
| | | .....E............ | (2/10) |
| | | ...............N.. | (1/10) |
| | | ...LG............. | (1/′10) |
| | | .................. | (1/10) |
| SEQ ID NO:15 | 96081 | QSPGGLDKGLSSL | (UNKNOWN MHC) |
| | Nef 21 | | |
| | | ...............P | (2/10) |
| | | .....R.......... | (1/10) |
| | | R......G....... | (1/10) |
| | | ...............F | (1/10) |
| | | .....E....D.... | (1/10) |
| | | .......G....... | (1/10) |
| | | ................ | (1/10) |

We claim:

1. A method of identifying at least one CTL-inducing epitope from at least one HIV protein, wherein the immune response directed against this epitope is capable of selecting for viral escape variants during the acute or peri-acute phase of infection, wherein the method comprises the steps of
a) examining the nucleic acid sequence encoding at least one HIV protein from at least one HIV-infected patient, wherein the sequence encoding the expressed protein is examined in the first 24 weeks after infection, to identify at least one region of the HIV protein that is variable as compared to the sequence of the protein at an earlier time point in infection, wherein the variable region indicates a CTL-inducing epitope, and
b) confirming that an immune response directed against the CTL-inducing epitope is capable of selecting for viral escape variants during the acute or periacute phase of HIV infection.

2. The method of claim 1 further comprising the step of testing peripheral blood mononuclear cells (PBMC) from HIV infected patients in the first six months after infection to confirm that CTL responses to the CTL-inducing epitope of step (a) are of high avidity.

3. The method of claim 1 wherein the sequence variation is detected between 0 and 24 weeks after infection.

4. The method in claim 1 wherein the HIV protein is selected from the group consisting of Gag, Env, Pol, Rev, Nef, Tat, Vpx, Vpu, and Vif.

5. The method of claim 1 wherein the identified epitope is examined for its ability to induce CD4⁺ helper T lymphocyte (HTL) cells.

6. The method of claim 1 wherein a minimal peptide needed to elicit the CTL response is determined.

7. The method of claim 1 wherein the confirmation of claim 1 is via a cellular assay selected from the group consisting of intracellular cytokine staining and ⁵¹Cr-release assays.

8. The method of claim 2 wherein the sequence variation is detected between 0 and 24 weeks after infection.

9. The method in claim 2 wherein the HIV protein is selected from the group consisting of Gag, Env, Pol, Rev, Nef, Tat, Vpx, Vpu, and Vif.

10. The method of claim 2 wherein the identified epitope is examined for its ability to induce CD⁴⁺ helper T lymphocyte (HTL) cells.

11. The method of claim 2 wherein a minimal peptide needed to elicit the CTL response is determined.

12. The method of claim 2 wherein the confirmation of claim 1 is via a cellular assay selected from the group consisting of intracellular cytokine staining and ⁵Cr-release assays.

13. A method of identifying at least one CTL-inducing epitope from an RNA virus, wherein an immune response directed against the epitope is capable of selecting for viral escape variants during the first 24 weeks of infection, wherein the method comprises the steps of
a) examining the viral nucleic acid sequences from virus-infected patients in the first 24 weeks after infection to identify at least one region of the virus that is variable, wherein the variable regions indicate a CTL-inducing epitope and
b) confirming that the epitope is capable of selecting for viral escape variants during the acute or periacute phase of viral infection.

14. The method of claim 13 wherein the confirmation of step (b) is via a cellular assay selected from the group consisting of intracellular cytokine staining and ⁵¹Cr-release assays.

15. The method of claim 13 further comprising the step of testing peripheral blood mononuclear cells (PBMC) from infected patients in the first six months after infection to confirm that CTL responses to the CTL-inducing epitope of step (a) are of high avidity.

16. The method of claim 13 wherein the minimal peptide needed to elicit the CTL response is determined.

17. The method of claim 15 wherein the minimal peptide needed to elicit the CTL response is determined.

18. A vaccine comprising a nucleic acid encoding at least one CTL-inducing epitope selected by the method of claim 1.

19. The vaccine of claim 18 comprising at least 2 CTL-inducing epitopes.

20. The vaccine of claim 18 comprising at least 3 CTL-inducing epitopes.

21. A vaccine comprising a nucleic acid encoding at least one CTL-inducing epitope selected by the method of claim 2.

22. The vaccine of claim 18 comprising at least 2 CTL-inducing epitopes.

23. The vaccine of claim 18 comprising at least 3 CTL-inducing epitopes.

24. A vaccine comprising a nucleic acid encoding at least one CTL-inducing epitope selected by the method of claim 13.

25. The vaccine of claim 24 comprising at least 2 CTL-inducing epitopes.

26. The vaccine of claim 24 comprising at least 3 CTL-inducing epitopes.

27. A vaccine comprising a nucleic acid encoding at least one CTL-inducing epitope selected by the method of claim 15.

28. The vaccine of claim 27 comprising at least 2 CTL-inducing epitopes.

29. The vaccine of claim 27 comprising at least 3 CTL-inducing epitopes.


SEQUENCE LISTING (15 sequences)

SEQ ID NO: 1 - protein, 8 aa, Simian immunodeficiency virus
TTPESANL

SEQ ID NO: 2 - protein, 8 aa, Simian immunodeficiency virus
STPESANL

SEQ ID NO: 3 - protein, 9 aa, Simian immunodeficiency virus
CTPYDINQM

SEQ ID NO: 4 - DNA, 20 bp, Simian immunodeficiency virus
tgatcctcgcttgctaactg

SEQ ID NO: 5 - DNA, 20 bp, Simian immunodeficiency virus
agcaagatggcgataagcag

SEQ ID NO: 6 - protein, 8 aa, Simian immunodeficiency virus
STPESANL

SEQ ID NO: 7 - protein, 9 aa, Simian immunodeficiency virus
YTSGPGIRY

SEQ ID NO: 8 - protein, 9 aa, Simian immunodeficiency virus
GLDKGLSSL

SEQ ID NO: 9 - protein, 9 aa, Simian immunodeficiency virus
CTYPDINQM

SEQ ID NO: 10 - protein, 11 aa, Simian immunodeficiency virus
ARRHRILDIYL

SEQ ID NO: 11 - protein, 9 aa, Simian immunodeficiency virus
KRQQELLRL

SEQ ID NO: 12 - protein, 8 aa, Simian immunodeficiency virus
GDYKLVEI

SEQ ID NO: 13 - protein, 8 aa, Simian immunodeficiency virus
STPESANL

SEQ ID NO: 14 - protein, 15 aa, Simian immunodeficiency virus
PDWQDYTSGPGIRYP

SEQ ID NO: 15 - protein, 13 aa, Simian immunodeficiency virus
QSPGGLDKGLSSL